(19) United States
(12) Patent Application Publication
(10) Pub. No.: US 2004/0191772 A1
(43) Pub. Date: Sep. 30, 2004

(54) Method of shuffling polynucleotides using templates

(76) Inventors: Daniel Marc Dupret, Calvisson (FR); Fabrice Lefevre, Nimes (FR); Jean Michel Masson, Toulouse (FR)

Correspondence Address: HUNTON & WILLIAMS LLP, INTELLECTUAL PROPERTY DEPARTMENT, 1900 K STREET, N.W., SUITE 1200, WASHINGTON, DC, 20006-1109, US

(21) Appl. No.: 10/360,783

(22) Filed: May 24, 2002

(51) Int. Cl. C12Q 1/68; C12M 1/34

(52) U.S. Cl. 435/6; 435/287.2

(57) ABSTRACT

Method of gene shuffling using hybridization of fragments on assembly templates, wherein the fragments are not themselves the templates. Invention is particularly aimed at generating novel polynucleotides that differ in some advantageous respect compared to a reference sequence. Invention further includes reaction mixtures created by or during the method, sequences created by the method, hosts and vectors containing same, and proteins translated therefrom.

### CROSS-REFERENCE TO RELATED APPLICATIONS

The following applications are hereby incorporated by reference in their entireties: U.S. application Ser. No. 09/840,861, filed Apr. 25, 2001; U.S. Provisional Application No. 60/285,978; U.S. application Ser. No. 09/723,316, filed Nov. 28, 2000; PCT Application No. PCT/FR99/01973, filed Dec. 8, 1999; French Patent Application No. FR98/10338, filed Dec. 8, 1998; the U.S. Application filed by Applicant on Apr. 25, 2002; and the PCT Application filed by Applicant on Apr. 25, 2002.

### BACKGROUND OF THE INVENTION

The present invention relates broadly to genetic recombination and to the field known variously as directed evolution, molecular breeding or DNA shuffling. The invention aims particularly at generating novel sequences with improved characteristics compared to those of a reference sequence. When performed outside a living organism, the process comprises a technique for in vitro evolution. The invention further relates to the sequences generated by the method, libraries of such sequences, hosts and vectors containing such sequences, proteins translated therefrom, to arrays that simulate the method of the invention, and to arrays in which the method can be performed. The invention further relates to intermediate products of the method, to reaction mixtures of certain types of polynucleotide fragments and assembly templates, and to compositions of certain assembly templates and recombinant polynucleotides produced therewith.

Various techniques are known to facilitate in vitro recombination of polynucleotide sequences. The most well-known conventional techniques are DNA shuffling with sexual PCR (multiple cycles with no added primer) and staggered extension (StEP), which both rely on polymerization.

Typically, in DNA shuffling with sexual PCR, DNase I randomly cuts polynucleotide sequences to form oligonucleotide fragments, the fragments initiate polymerization or PCR extension, and the recombined polynucleotides are amplified. At each hybridization step, crossovers occur at homologous regions among the sequences (“strand switching”). A schematic representation of this method appears in FIG. 1A.

StEP consists of mixing various polynucleotide sequences containing various mutations in the presence of a pair of initiators. Hybridization of the initiators and polymerization is consolidated into a single, very brief step. These conditions make it possible to hybridize the initiators but also slow the polymerization so that the initiators have time to synthesize only fragments which, after denaturation, re-hybridize randomly to the various polynucleotide sequences. A schematic representation of this method appears in FIG. 1B.

Relying heavily on polymerization has drawbacks. Such methods do not confer control over the rate or location of recombination, which occurs randomly during the successive stages of polymerization. Depending on the conditions and polymerase used, the polymerization can also produce either undesired supplemental mutations or insufficient numbers of mutations. The latter occurs when long gaps are filled with residues that are fully complementary to the opposite strand. Further, after enough cycles, the fragments grow very long and become what are known as “mega-initiators” (6). Mega-initiators can cause various problems, particularly when the starting polynucleotides exceed about 1.5 kb.

### ADVANTAGES OF THE INVENTION

The invention need not rely on polymerization, size fractionation (isolation of fragments by size) or amplification of the polynucleotides or fragments. Further, Applicant believes, though not wishing except where stated otherwise to be limited thereto in any way, that the invention and embodiments confer broad advantages.

First, the invention provides some control over the locations of recombination. Hybridization on a template, especially without polymerization, enables precise control of the locations where recombination occurs. For example, if a target protein contains an active site that one desires to leave unchanged, the invention is capable of limiting recombination to regions other than the active site. Furthermore, the invention can achieve high recombination between closely neighboring sequence segments. Rather than treating close-lying sequences as “linked,” and moving them in chunks, the invention can separate the close-lying sequences. Therefore, in a sense the invention also achieves high resolution, fidelity and quality of genetic diversity. Indeed, the embodiment of the invention that employs nonrandom fragmenting can use fragments as short as about 15 residues.

The invention may also generate more recombination and incorporation of fragments per reaction cycle, particularly in embodiments other than ligation-only embodiments (defined below). In other words, it achieves a high quantity of genetic diversity. High quantity is achieved directly by stimulating more total recombination events. It is achieved indirectly by increasing overall efficiency. Overall efficiency is increased by using, inter alia, oriented ligation. Without oriented ligation, a sequence cut into “n” fragments will reassociate into an enormous variety of possible forms, even if only one or a few forms are useful. The present invention, on the other hand, facilitates direct achievement of the desired form. Indeed, in some embodiments of the invention, it is possible to obtain a recombinant polynucleotide after only a single reaction cycle.

Typically, the invention further increases efficiency by generating relatively few unshuffled parental clones and duplicate chimeras. Avoiding these unwanted by-products provides room for more novel chimeras. The conventional methods may produce screening libraries that consist of 30% to 70% parental DNA. In all methods of directed evolution, molecular breeding or gene shuffling, a screening library of recombinant DNA molecules is produced and these molecules are expressed and screened. Screening is the most expensive and time-consuming part of the process since the libraries may contain 100,000 to several million recombinant molecules. Eliminating parental DNA from the screening libraries mitigates this problem. The elimination of parental DNA is enhanced when the template is transient, as in more preferred embodiments of the invention, because the final population is composed of only the new, variant polynucleotides.

Preferred embodiments of the method, particularly those that employ solitary-stranded or non-identical templates or fragments, also facilitate low-homology shuffling, e.g., of distantly-related members of gene families. The terms “solitary-stranded” and “non-identical” are used herein to describe a population of particular single-stranded sequences that do not complement each other because they are all from the same strand, either the sense or antisense strand, of one polynucleotide or multiple homologous polynucleotides. Since on-identical fragments, for example, are not complementary or at least not strictly complementary to another fragment in the reaction mixture, hybridization is not biased toward the “wild type” sequences that would be formed by complementary fragments. Hybridization temperatures can be adjusted to the degree of homology among the sequences, thereby maximizing diversity and greatly increasing the chances of finding the right mutant in the shortest number of recombination cycles. (Note that the invention may still comprise achieving a desired bias, e.g., by using higher amounts of one parental polynucleotide.)

In addition, the invention demands little preparation of the starting DNA library. The invention allows immediate use of complex or genomic DNA which may include introns. Some other methods require time-consuming isolation of mRNA and re-creation of the cDNA sequence in order to generate fragments for shuffling or reassembly.

Additional advantages of the invention or its embodiments are further described herein.

### SUMMARY OF THE INVENTION

Although the present invention relates broadly to genetic recombination, “recombination” is somewhat of a misnomer with regard to the invention insofar as the term implies that two strands disassociate and then recombine with each other to form a recombinant sequence. In other words, the invention does not rely on strand switching or crossovers. Nevertheless, “recombination” and related terms are retained herein, subject to this caveat.

In one embodiment, the invention includes a template-mediated method for shuffling polynucleotides, comprising hybridizing fragments of at least two homologous polynucleotides to one or more assembly templates to form at least one recombinant polynucleotide, wherein the fragments are shorter than all or substantially all of the assembly templates.

In a preferred embodiment, the assembly template is not fragmented. In a more preferred embodiment, no polymerization or extension is used to create a sequence complementary to the template or to fill in long gaps. Similarly, in a preferred embodiment, the fragments are non-initiating fragments that do not act as extension primers. In yet another preferred embodiment, the formation of the recombinant polynucleotide entails (i) ligating nicks, and (ii) where necessary, any one of or any combination of the following gap filling techniques:

filling in gaps by further hybridizing said fragments to said templates to increase the number of fragments that are adjacently hybridized,

filling in short gaps by trimming any overhanging flaps of any partially hybridized fragments, and

filling in short gaps via polymerization.

In a yet a more preferred embodiment, no polymerization is used except to optionally amplify the final recombinant polynucleotides. In a still more preferred embodiment, no polymerization is used at all. Most preferably, the method uses only a ligase and/or flap endonuclease to form the hybridized fragments into a recombinant polynucleotide.

Preferably, any of the steps or substeps may be repeated as necessary. In another embodiment, the method of the invention generates a recombinant polynucleotide after only one round, cycle or single operation of each step of the invention. In a preferred embodiment, the method further comprises step (d) selecting at least one of said recombinant polynucleotides that has a desired property. More preferably, the steps occur in vitro (outside a living organism). In some preferred embodiments, the method employs, inter alia, nonrandom fragmentation, transient templates, and non-identical templates or fragments. In a more preferred embodiment, the assembly template is devised.

In an alternative embodiment, at least two of the fragments adjacently hybridize to the template, more preferably all of the fragments adjacently hybridize.

In another alternative embodiment, the invention comprises a template-mediated method for nonrandom low-homology shuffling of gene families in vitro. Whether homology is considered low differs in different contexts, but homology that ranges below 50% (e.g., 40-70% or 20-45%) would typically be considered low. In another alternative embodiment, the parental polynucleotides vary in length by more than two residues.

In yet another alternative embodiment, the invention comprises a template-mediated method for in vitro nonrandom shuffling of mutation-containing zones of polynucleotide alleles. This embodiment further comprises locating restriction sites for mutation-containing zones among the alleles, and obtaining fragments corresponding to those restriction sites.

The invention further includes sequences created by the method, libraries of same, hosts and vectors containing same and proteins translated therefrom. It also includes a logical array, such as a computer algorithm, that simulates the inventive method, or a physical array, such as a biochip, in which the inventive method may be performed. The invention further relates to intermediate products of the method, to reaction mixtures of polynucleotide fragments and assembly templates that can be used to carry out some or all steps of the method, and to compositions of certain assembly templates and recombinant polynucleotides produced therewith.

The foregoing summaries are nonexhaustive. Further alternative embodiments and additional optional features of the invention appear throughout this application.

### DEFINITIONS

“In vitro”, as used herein, refers to any location outside a living organism.

“Homologous” polynucleotides differ from each other at least at one corresponding residue position. Thus, as used herein, “homologous” encompasses what is sometimes referred to as “partially heterologous.” The homology, e.g., among the parental polynucleotides, may range from 20 to 99.99%, preferably 30 to 90, more preferably 40 to 80%. In some embodiments the term homologous may describe sequences that are, for example, only about 2045% identical at corresponding residue positions. Homologous sequences may or may not share with each other a common ancestry or evolutionary origin.

“Polynucleotide” and “polynucleotide sequence” refer to any nucleic or ribonucleic acid sequence, including mRNA, that is single-stranded, non-identical or partially or fully double-stranded. When partially or fully double-stranded, each strand may be identical or heterologous to the other, unless indicated otherwise. A polynucleotide may be a gene or a portion of a gene. “Gene” refers to a polynucleotide or portion thereof associated with a known or unknown biological function or activity. A gene can be obtained in different ways, including extraction from a nucleic acid source, chemical synthesis and synthesis by polymerization. “Parental polynucleotide” and “parent” are interchangeable synonyms that refer to the polynucleotides that are fragmented to create donor fragments. Parental polynucleotides are often derived from genes. “Recombined polynucleotide,” “mutant polynucleotide,” “chimeric polynucleotide” and “chimera” generally refer to the polynucleotides that are generated by the method. However, these terms may refer to other chimeric polynucleotides, such as chimeric polynucleotides in the initial library. “Reference sequence” refers to a polynucleotide, often from a gene, having desired properties or properties close to those desired, and which is used as a target or benchmark for creating or evaluating other polynucleotides.

“Polynucleotide library” and “DNA library” refer to a group, pool or bank of polynucleotides containing at least two homologous polynucleotides or fragments thereof. A polynucleotide library may comprise either an initial library or a screening library. “Initial library,” “initial polynucleotide library,” “initial DNA library,” “parental library” and “start library” refer to a group, pool or bank of polynucleotides or fragments thereof containing at least two homologous parental polynucleotides or fragments thereof. The initial library may comprise genomic or complex DNA and include introns. It may also comprise sequences generated by prior rounds of shuffling. Similarly, a screening library or other limited library of recombinant polynucleotides or fragments may serve as and be referred to as an initial library. “Screening library” refers to the polynucleotide library that contains chimeras generated by the inventive process or another recombinant process.

“Residue” refers to an individual nucleotide or ribonucleotide, rather than to multiple nucleotides or ribonucleotides. Residue may refer to a free residue that is not part of a polynucleotide or fragment, or to a single residue that forms a part of a polynucleotide or fragment.

“Donor fragments” and “fragments” generally refer to the fragmented portions of parental polynucleotides. Fragments may also refer to supplemental or substitute fragments that are added to the reaction mixture and/or that derive from a source other than fragmentation of the parental polynucleotides. Most or all of the fragments should be shorter than the parental polynucleotides. Most or all of the fragments are shorter than the assembly templates. As used herein, the donor fragments preferably do not initiate polymerase extension, i.e., they are not primers.

“Nonrandom” and “controlled,” as used herein, refer broadly to the control or predictability, e.g., over the rate or location of recombination, achieved via the template and/or ligation-orientation of the invention. Nonrandom and controlled may also refer more specifically to techniques of fragmenting polynucleotides that enable some control or predictability over the size or sequence of the resulting fragments. For example, using restriction enzymes to cut the polynucleotides provides some control over the characteristics of the fragments. Note that the invention may still be considered nonrandom when it employs random fragmentation (typically by DNase I digestion). In such cases, the assembly template and other features of the invention still provide a degree of control. In preferred embodiments, however, the fragmentation is nonrandom or controlled.

“Assembly template” and “template” refer to a polynucleotide used as a scaffold or matrix upon which fragments may anneal or hybridize to form a partially or fully double-stranded polynucleotide. The templates of the invention are to be distinguished from various sequences in the art that have been referred to as “templates.” For example, the templates of the present invention do not include overlapping donor fragments that facilitate the extension of complementary donor fragments hybridized thereto. As such, the template is distinct from the donor fragments at some point in the process. The templates of the present invention also do not include those sequences used in processes that rely heavily on polymerase extension to generate all or most of the opposing strand. In other words, the invention relies on hybridization of donor fragments to form the brunt of the recombinant strand. Preferably, the template strand of the recombinant polynucleotide formed by the process, although it may itself be recombinant, does not undergo recombination during the process. In other words, preferably no donor fragments are incorporated into the template strand during a cycle of the process. The template may be synthetic, result from shuffling or other artificial processes, or it may exist in nature. “Transient template” refers to a template that is not itself incorporated into the final recombinant polynucleotides. This transience is caused by separation or disintegration of the template strand of the nonfinal recombinant polynucleotide generated during the method. The template may derive from the reference sequence, the initial library, the screening library or elsewhere. Although the template may comprise or derive from a parental polynucleotide of the initial library, in a preferred embodiment the template is “devised,” and a polynucleotide does not qualify as a devised template if it enters the shuffling process accidentally, e.g., by somehow slipping into the hybridization step without being fragmented. In other words, a devised template is not entirely random or accidental. Rather, at least to some extent a devised template is directly or indirectly obtained for use as a template by a human being, or a computer operated thereby, via purposeful planning, conception, formulation, creation, derivation and/or selection of either a specific desired polynucleotide sequence(s) or a sequence(s) from a source(s) that is likely to contain a desired sequence(s). Finally, note that the word “template” is unnecessary. A polynucleotide, often a single-stranded polynucleotide, that acts like the template of the invention is indeed the template of the invention whether or not it is referred to as a template. “Matrix” or “scaffold” are also synonyms of template. Similarly, embodiments of the method are “template-mediated” whether or not they are expressly described as such. For example, the “ligation-oriented” and “exonuclease-mediated” embodiments of the invention use the template of the invention.

“Solitary-stranded” or “non-identical” is used to describe a population of single-stranded sequences that do not complement each other because they are all from the same strand, either sense or antisense, of one polynucleotide or multiple homologous polynucleotides. In other words, sequences from the opposing complementary strands are absent, so the population contains no sequences that are complementary to each other. For example, the population of non-identical fragments may consist of fragments of the top strands of the parental polynucleotides, whereas the population of non-identical templates may consist of bottom strands of one or more of the parental polynucleotides.

“Ligation” refers to creation of a phosphodiester bond between two residues.

“Nick” refers to the absence of a phosphodiester bond between two residues that are hybridized to the same strand of a polynucleotide. Nick includes the absence of phosphodiester bonds caused by DNases or other enzymes, as well as the absences of bonds between adjacently hybridized fragments that have simply not been ligated. As used herein, nick does not encompass residue gaps.

“Gap” and “residue gap,” as used herein, refer to the absence of one or more residues on a strand of a partially double-stranded polynucleotide. In some embodiments of the invention, short gaps (less than approximately 15-50 residues) are filled in by polymerases and/or flap trimming. Long gaps are conventionally filled in by polymerases.

“Hybridization” has its common meaning except that it may encompass any necessary cycles of denaturing and re-hybridization.

“Adjacent fragments” refer to hybridized fragments whose ends are flush against each other and separated only by nicks, not by gaps.

“Ligation-only” refers to embodiments of the invention that do not utilize or require any gap filling, polymerase extension or flap trimming. In ligation-only embodiments, all of the fragments hybridize adjacently. Note that embodiments that are not ligation-only embodiments still use ligation.

As used herein, “ligation-oriented,” “oriented ligation” and “ligation-compatible” generally represent or refer to a template-mediated process that enables ligation of fragments or residues in a relatively set or relatively predictable order. In “ligation-only” embodiments, the method employs no gap filling techniques and instead relies on ligation of adjacent fragments, often achieved after multiple hybridization events.

As used herein, “exonuclease-mediated” generally refers to a template-mediated process that employs flap trimming to enable ligation of fragments or residues in a relatively set or relatively predictable order.

### BRIEF DESCRIPTION OF THE DRAWINGS

Reference is made to the appended drawings in which:

FIG. 1 is a schematic representation of conventional DNA-shuffling (FIG. 1A) and StEP (FIG. 1B).

FIG. 2 is a schematic representation of an embodiment of the process of the invention and of certain of its variations and applications.

FIG. 3 represents the positions of the ten zones of mutations (Pvu II and Pst I) carried by each mutant of the ponB gene.

FIG. 4 represents the position of the primers used compared to the sequence of the ponB gene.

FIG. 5 represents the migration on agarose gel of RLR and of PCR reaction products of these RLR reactions.

FIG. 6 represents the position of the mutations compared to the restriction fragments.

FIG. 7 depicts the results of error-prone PCR on WT XynA gene using 1% agarose gel.

FIG. 8 depicts thermal inactivation of mutant 33 at 82° C.

FIG. 9 depicts the results of fragmentation of PCR products with six restriction endonucleases, using 3% agarose gel.

FIG. 10 depicts the results of L-Shuffling™ experiments using 1% agarose gel.

FIG. 11 depicts the results of using PCR Pfu on L-Shuffling™ products, using 1% agarose gel.

FIG. 12 depicts thermal inactivation of mutants at 95° C.

FIG. 13 depicts the results of DNaseI fragmentation of Thermotoga neapolitana (A) and Acidobacterium capsulatum (B) genes, using 1% agarose gel.

FIG. 14 depicts the results of L-Shuffling™ experiments, using 1% agarose gel.

FIG. 15A depicts the results of L-Shuffling™ using n cycles of steps (b) and (c), and FIG. 15B shows the PCR amplification of the corresponding L-Shuffling™ products.

FIG. 16 depicts the results of L-Shuffling™ experiments using increased quantities of fragments.

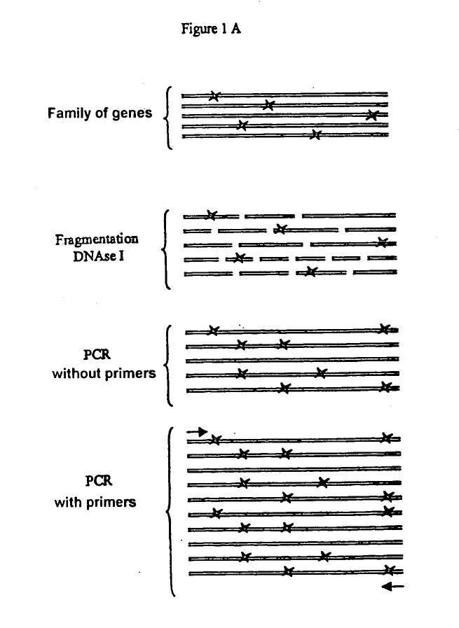

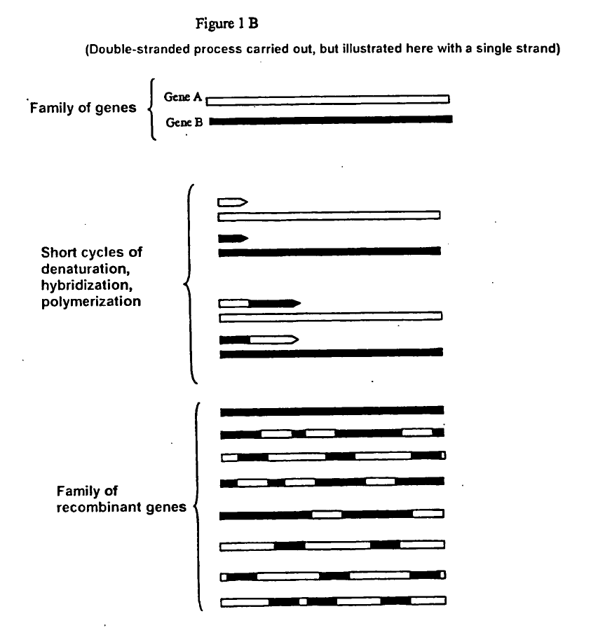

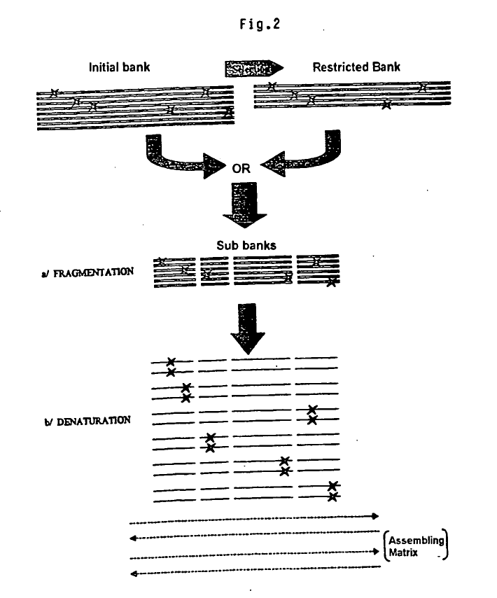

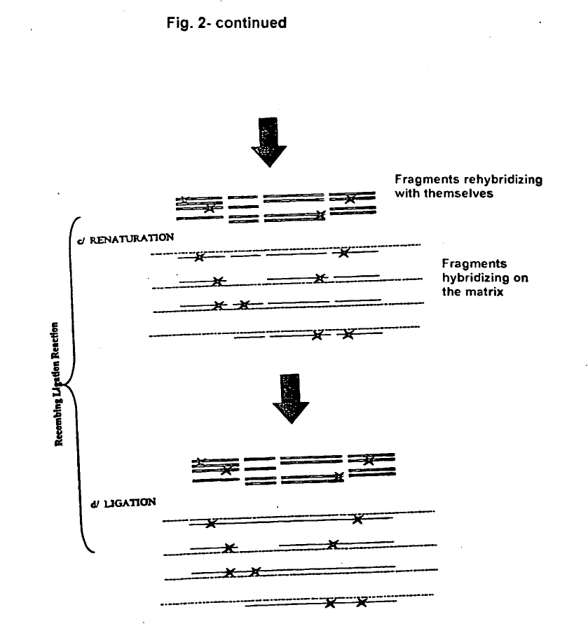

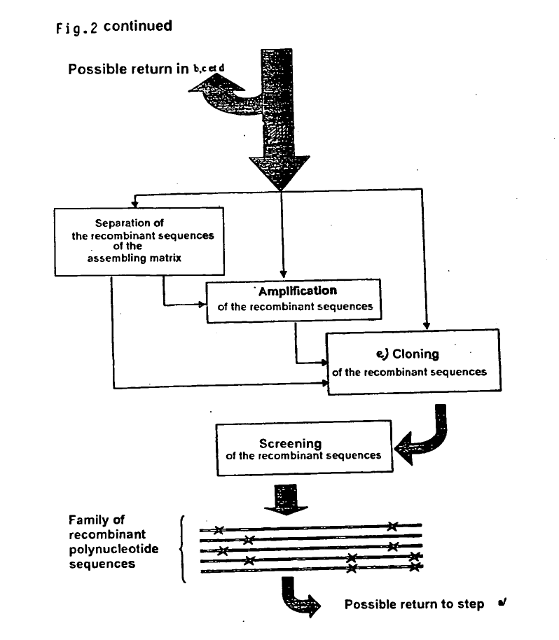

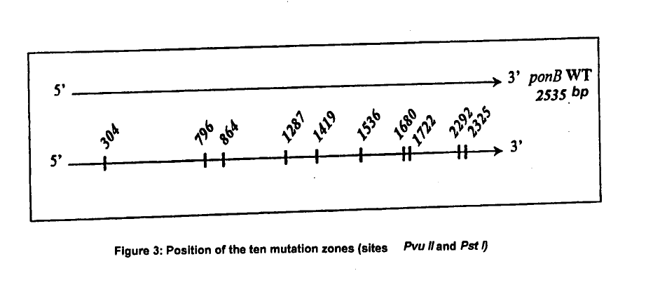

### DETAILED DESCRIPTION OF PREFERRED EMBODIMENTS OF THE INVENTION

One embodiment of the invention comprises a template-mediated method for shuffling polynucleotides, comprising hybridizing fragments of at least two homologous polynucleotides to one or more assembly templates to form at least one recombinant polynucleotide, wherein the fragments are shorter than all or substantially all of the assembly templates.

Preferably, once the partially double-stranded polynucleotides become adequately double-stranded, they are selected for advantageous properties compared to those of one or several reference sequences. Advantageous characteristics may include, for example, thermostability of an enzyme or its activity under certain pH or salinity conditions. Among many other possible uses, such enzymes may be used for desizing textile fibers, bleaching paper pulps, producing flavors in dairy products, or biocatalyzing synthesis of new therapeutic molecules.

The process may also comprise disintegrating the template strand or separating it from the recombinant strand before or after the selection. It may further comprise amplifying the recombinant sequences before selection, or cloning of recombinant polynucleotide sequences after separation of the recombinant strand from the template. Any amplification technique is acceptable. Due to initiators that can hybridize only to the ends of recombinant sequences, PCR enables selective amplification of the recombinant sequences. However, unlike shuffling with sexual PCR, the invention does not require amplification during the recombination reactions.

A preferred screening technique entails in vitro expression via in vitro transcription of recombinant polynucleotides, followed by in vitro translation of the mRNAs. This technique eliminates cellular physiological problems and the drawbacks connected with in vivo expression cloning. Further, this technique is easily automated, which enables screening of a high number of recombinant sequences.

Although embodiments of the invention may not in fact need to cycle through any steps more than once (“non-iterative”), the invention also encompasses repetition of any of its steps or substeps. For instance, the process may or may not entail multiple hybridization events. The hybridization may encompass any necessary cycles of denaturing and re-hybridizing. If necessary, repeated hybridization may be performed in part or in whole on ligated and/or non-ligated fragments produced during the process, rather than only on the initial donor fragments produced. The ligation-only embodiments typically require multiple iterations. In addition to encompassing repetition of steps, the invention includes embodiments that allow simultaneous operation of those steps that are known in the art as capable of simultaneous operation.

In a preferred embodiment, the initial library is itself produced by the present invention. Either in vivo or in vitro screens can be used to form this library for repeating the process of the invention. The recombinant sequences selected after a first running of the process can be optionally mixed with other sequences.

The initial library can also be produced by any method known to one skilled in the art, for example, by starting from a wild-type gene, by successive managed stages of mutagenesis, by “error-prone” PCR (2), by random chemical mutagenesis, by random mutagenesis in vivo, or by combining genes of close or relatively distant families within the same or different species. Preferably, the initial library results from chain polymerization reactions under conditions that create random, localized mutations. The invention may also comprise synthetic sequences.

Assembly Templates

The assembly template is, for example, a polynucleotide from the initial library or a polynucleotide produced therefrom. The template may be synthetic, result from shuffling or other artificial processes, or it may exist in nature. The template can be single- or double-stranded. If double-stranded, it must be denatured before actual hybridization can occur.

Preferred embodiments use a non-identical template. More preferred embodiments use as a non-identical template the bottom-strand from one parent polynucleotide and use as fragments top-strand fragments from other homologous parents. This prevents re-annealing of sequences to their own complementary strands. To obtain non-identical DNA molecules, a Bluescript phagemide or a vector of the family of filamentous phages such as M13 mp18 can be used. Another method consists in creating double-stranded molecules by PCR by using an initiator phosphorylated at 5′ and the other non-phosphorylated. The digestion of the lambda phage by the exonuclease will destroy the strands of DNA phosphorylated at 5, leaving the non-phosphorylated strands intact. Another method of creating non-identical molecules consists in making an amplification, by asymmetric PCR, starting from a methylated DNA template. Digestion by Dpn I will destroy the methylated strands, leaving intact the amplification products that will then be able to be purified after denaturation.

Preferred embodiments also use transient templates that are not incorporated within the final recombined polynucleotide, e.g., not part of the polynucleotide that is transferred to the screening library. One technique of conferring transience employs markers on either the recombinant strand or the template. For example, the template may be marked by a hapten and separated by, for example, fixing an antihapten antibody on a carrier or by initiating a biotin-streptavidin reaction. Another technique comprises synthesizing a transient template by PCR amplification using methylated dATP, which enables degradation of the template by restriction endonuclease Dpn I. In this case, the recombinant strand must not contain methylated dATP. A transient template can also by prepared by PCR amplification with dUTP, which enables degradation with uracil-DNA-glycosylase. Conversely, it is possible to protect the recombinant strand by amplifying it with selective PCR with oligonucleotides carrying phosphorothioated groups at 5′. A treatment with an exonuclease thus enables exclusive degradation of the template. In most preferred embodiments, transience is conferred by using a uracil-containing template such as mRNA. mRNA has a higher affinity of binding and can be removed by mRNA-specific enzymes. Such an mRNA template can be prepared in vivo or in vitro. In more preferred embodiments, use of an mRNA template entails including in the process at least three primers linked with a ligase.

In yet another preferred embodiment, the template enables orientation of multimolecular ligation of flush ends. In this embodiment, the template comprises a single- or double-stranded polynucleotide that is exactly complementary to the 3′ end of a first fragment and to the 5′ end of a second fragment that is adjacent to the first fragment in the parental polynucleotide. This facilitates adjacent hybridization of these two ends on the template.

Further embodiments include any or all of the following: the template and donor fragments are from different sources, the template is separately added to the reaction mixture, and/or the template is modified in specific ways to increase chimeragenesis.

Donor Fragments

Fragments can be recruited from homologous polynucleotides, related genes or from other genes. The parental DNA need not be characterized at all, but can be extracted from cells, clinical samples or the environment. As used herein, “hybridizing fragments” encompasses not only using pre-fragmented single- or double-stranded fragments from an initial fragment-containing library, but also the substep of fragmenting single- or double-stranded parental polynucleotides from an initial library to obtain the fragments which are then hybridized. he fragments may comprise fragments produced by combining distinct libraries of fragments, fragmenting parental polynucleotides from distinct starting libraries or fragmenting parental polynucleotides from the same library in different ways, such as with different restriction enzymes. Furthermore, the invention may comprise employing more fragments from one parental polynucleotide than another. For example, an experimenter using the process may bias the results by using more fragments of or parts of polynucleotide X than fragments of or parts of polynucleotide Y.

In one embodiment, supplemental single- or double-stranded fragments of variable length are added to the reaction mixture. These supplemental fragments may substitute for some of the donor fragments, particularly if their sequences are homologous to the donor fragments. Such supplemental fragments may, for example, introduce one or more direct mutations. Donor or supplemental fragments may also comprise synthetic fragments.

Fragmenting may occur before or after denaturing of the sequences that are fragmented. Fragmentation can be controlled or random. If random, any enzymatic or mechanical means known to those skilled in the art can be used to randomly cut the DNA, for example, digestion by DNase I or ultrasonication. If the fragmentation is controlled, it facilitates management over the degree, rate, efficiency and/or location of recombination. A preferred embodiment comprises hydrolyzing the parental polynucleotides with restriction enzymes to create restriction donor fragments. Restriction enzymes provide control over the degree, rate and efficiency of recombination by controlling the number of fragments produced per sequence. For example, the number may be increased by using restriction enzymes with many cutting sites or by using several different restriction enzymes. The greater the number of fragments produced per sequence, the greater the number (n) of fragments that must be recomposed to form a recombinant sequence. Preferably, n is 3 or more.

By controlling the nature and position of the fragment ends, restriction enzymes further provide control over not only degree and rate but also the location where recombination occurs. For example, the fragmenting can be designed so that the cuts occur in zones of the parent sequences that are homologous to zones in a reference sequence or an assembly template.

Fragments are preferably about 15-500 residues in length. When fragmentation is performed nonrandomly, the fragments are advantageously at least 15 residues in length and more preferably about 15-40 residues in length. The phrase “at least 15 residues” means between about 15 residues and the length of the longest polynucleotide used less one residue. When fragmentation is performed randomly, they are more preferably about 50-500 residues in length.

Preferably, the ends of at least two of the fragments are capable of being adjacently hybridized and ligated. In a preferred embodiment the invention employs flap trimming enzymes to make ligatable ends that would otherwise result in unproductive fragments. These enzymes recognize and degrade or cut in a specific way the nonhybridized ends of fragments when they cover other hybridized fragments on the same template.

A preferred enzyme is Flap endonuclease. When the fragments are initially double-stranded, an embodiment of the invention comprises using specific exonucleases that recognize and degrade single-stranded sequences like the nonhybridized ends of the fragments. Such single-strand exonucleases or Flap endonucleases are preferably at a concentration (e.g., about 1.8-2.2 μg/ml of Flap endonuclease) that avoids their more general exonuclease activity, which could, for example, degrade the templates or recombinant sequences. These enzymes increase the number of fragment ends that can be ligated, which is particularly useful for randomly cut fragments because they tend to result in many overhanging flaps. Use of such enzymes with low hybridization temperatures and/or high hybridization times (e.g., two minutes) also facilitates recombination between low-homology polynucleotides. For example, a preferred embodiment that employs random fragmenting includes use of a Flap endonuclease and a wide range of hybridization temperature (e.g., from 5 to 65° C.) that can be disconnected from ligation with regard to temperature, particularly when the hybridization temperature is lower than the high ligation temperature (e.g., about 60-75° C.). Most preferably, the Flap endonuclease concentration is about 2 μg/ml, the hybridization temperature is about 10° C. and the ligation termperature is about 65° C. When such trimming enzymes are employed, they are preferably thermoresistant, thermostable and active at high temperatures, like the ligase.

Alternative Embodiments and Optional Features of the Invention

Unlike conventional shuffling methods, various embodiments of the invention do not require thermocycling, e.g., the repeated heating and cooling necessary for sexual PCR. In various embodiments, the process may be used to create gene-length polynucleotides or short polynucleotides. In various embodiments, hybridization may occur under conditions of low stringency. In various embodiments, the ratio between templates and chimeric polynucleotides produced is about 1. In various embodiments, no DNases are employed. In various embodiments, the initial library comprises variants of a single gene. In various embodiments, the initial library may comprise polynucleotides having artificially induced point mutations. In various embodiments, the invention may be used for whole genome shuffling. In various embodiments, the steps may occur in vivo rather than in vitro. Further, when amplifying fragments by PCR, for example, the initiated sequences can be designed to produce fragments whose ends are adjacent all along the assembly template.

Additional alternative embodiments of the invention are listed below. This list is nonexhaustive and variations of these embodiments may appear in the claims and elsewhere in this application.

A polynucleotide shuffling reaction mixture comprising fragments of at least two homologous polynucleotides and at least one assembly template upon which the fragments can hybridize, wherein the fragments are shorter than all or substantially all of the templates.

A polynucleotide shuffling reaction mixture comprising free fragments of at least two homologous polynucleotides and at least one partially double-stranded polynucleotide comprising a strand of an assembly template and an opposite partial strand of hybridized fragments, wherein the free fragments are shorter than all or substantially all of the templates.

A method for producing a recombinant DNA encoding a protein, the method comprising: (a) digesting at least a first and second DNA substrate molecule, wherein the at least first and second substrate molecules are homologous and differ from each other in at least one nucleotide, with a restriction endonuclease, wherein the at least first and second DNA substrate molecules each encode a protein, or are homologous to a protein-encoding DNA substrate molecule; (b) ligating the resulting mixture of DNA fragments to generate a library of recombinant DNA molecules, which library comprises a plurality of DNA molecules, each comprising a subsequence from the first nucleic acid and a subsequence from the second nucleic acid, wherein the plurality of DNA molecules are homologous; (c) screening or selecting the resulting products of (b) for a desired property; (d) recovering a recombinant DNA molecule encoding an evolved protein; and (e) repeating steps (a)-(d) using the recombinant DNA molecule of step (d) as the first or second DNA substrate molecule of step (a), whereby a recombinant DNA encoding a protein is produced. Preferably, steps (a)-(d) are repeated more than once. More preferably, the first or second DNA substrate molecule comprises a gene cluster. Still more preferably, at least one restriction endonuclease fragment from a DNA substrate molecule is isolated and subjected to mutagenesis to generate a library of mutant fragments. The library of mutant fragments may be used in the ligation of (b). Even more preferably, the mutagenesis comprises recursive sequence recombination. The product of (d) may also be subjected to mutagenesis, preferably recursive sequence recombination. Further, the product of (e) may be used as a DNA substrate molecule in (b). Also, the recombinant DNA substrate molecule of (d) may comprise a library of recombinant DNA substrate molecules. Some other preferred features of this alternative embodiment appear elsewhere in this application.

A method for making recombined nucleic acids, the method comprising: (a) providing at least one single-stranded polynucleotide; (b) providing one or more nucleic acids, at least one of which differs from the single-stranded polynucleotide(s) in at least one nucleotide, and fragmenting the one or more nucleic acids to produce a plurality of non-identical nucleic acid fragments that are capable of hybridizing to the single-stranded polynucleotide(s); (c) contacting the single-stranded polynucleotide(s) with the plurality of nucleic acid fragments, thereby producing annealed nucleic acid products; (d) contacting the products of (c) with a polymerase; and, (e) contacting the products of (d) with a ligase, thereby producing recombined nucleic acids annealed to the single-stranded polynucleotide(s). Preferred features of this alternative embodiment appear elsewhere in this application.

A method for making a modified or recombinant nucleic acid, the method comprising: (a) providing a selected single-stranded template nucleic acid; (b) contacting the selected single-stranded template nucleic acid with a population of nucleic acid fragments, wherein the population of nucleic acid fragments comprises one or more of: (i) nucleic acid fragments which comprise nucleic acid sequences which are homologous to the single-stranded template nucleic acid; (ii) nucleic acid fragments resulting from digestion of at least first substrate molecules with a DNase, (iii) nucleic acid fragments which comprise nucleic acid sequences produced by mutagenesis of a parental nucleic acid, (iv) nucleic acid fragments comprising at least one nucleic acid sequence which is homologous to the single-stranded template nucleic acid, which sequence is present in the population at a concentration of less than 1% by weight of the total population of nucleic acid fragments, (v) nucleic acid fragments comprising at least ˜one-hundred nucleic acid sequences which are homologous to the template, or (vi) nucleic acid fragments comprising sequences of at least 50 nucleotides, thereby producing an annealed nucleic acid product; and (c) contacting the annealed nucleic acid with a polymerase and a ligase, thereby producing a recombined nucleic acid strand, wherein the template nucleic acid comprises uracil and the method further comprises degrading the template nucleic acid. Some preferred features of this alternative embodiment appear elsewhere in this application.

A method for making a recombined nucleic acid, the method comprising: (a) providing a selected single-stranded template nucleic acid; (b) contacting the selected single-stranded template nucleic acid with a population of nucleic acid fragments, wherein the population of nucleic acid fragments comprises one or more of: (i) nucleic acid fragments which comprise nucleic acid sequences which are homologous to the single-stranded template nucleic acid; (ii) nucleic acid fragments resulting from digestion of at least first substrate molecules with a DNase, (iii) nucleic acid fragments which comprise nucleic acid sequences produced by mutagenesis of a parental nucleic acid, (iv) nucleic acid fragments comprising at least one nucleic acid sequence which is homologous to the single-stranded template nucleic acid, which sequence is present in the population at a concentration of less than 1% by weight of the total population of nucleic acid fragments, (v) nucleic acid fragments comprising at least one hundred nucleic acid sequences which are homologous to the template, or (vi) nucleic acid fragments comprising sequences of at least 50 nucleotides, thereby producing an annealed nucleic acid product; and (c) contacting the annealed nucleic acid with a polymerase and a ligase, thereby producing a recombined nucleic acid strand, wherein the template nucleic acid comprises uracil and the method further comprises degrading the template nucleic acid and releasing the resulting cleaved template nucleic acid from the annealed nucleic acid. Some preferred features of this alternative embodiment appear elsewhere in this application.

A method for making a recombined nucleic acid, the method comprising: (a) providing a selected single-stranded template nucleic acid; (b) contacting the selected single-stranded template nucleic acid with a population of nucleic acid fragments, wherein the population of nucleic acid fragments comprises one or more of: (i) nucleic acid fragments which comprise nucleic acid sequences which are homologous to the single-stranded template nucleic acid; (ii) nucleic acid fragments resulting from digestion of at least first substrate molecules with a DNase, (iii) nucleic acid fragments which comprise nucleic acid sequences produced by mutagenesis of a parental nucleic acid, (iv) nucleic acid fragments comprising at least one nucleic acid sequence which is homologous to the single-stranded template nucleic acid, which sequence is present in the population at a concentration of less than 1% by weight of the total population of nucleic acid fragments, (v) nucleic acid fragments comprising at least one hundred nucleic acid sequences which are homologous to the template, or (vi) nucleic acid fragments comprising sequences of at least 50 nucleotides, thereby producing an annealed nucleic acid product; (c) contacting the annealed nucleic acid with a polymerase and a ligase, thereby producing a recombined nucleic acid strand; and (d) transforming the recombined nucleic acid into a host, wherein the host is a mutS host. Some preferred features of this alternative embodiment appear elsewhere in this application.

A method of isolating nucleic acid fragments from a set of nucleic acid fragments, the method comprising: hybridizing at least two sets of nucleic acids, wherein a first set of nucleic acids comprises single-stranded nucleic acid templates and a second set of nucleic acids comprises at least one set of nucleic acid fragments; separating the hybridized nucleic acids from nonhybridized nucleic acids by at least one first separation technique; and, denaturing the separated hybridized nucleic acids to yield the single-stranded nucleic acid templates and isolated nucleic acid fragments. Some preferred features of this alternative embodiment include: the first set of nucleic acids comprises nucleic acids selected from the group consisting of sense cDNA sequences, antisense cDNA sequences, sense DNA sequences, antisense DNA sequences, sense RNA sequences, and antisense RNA sequences; the first and second sets of nucleic acids comprise substantially homologous sequences; the second set of nucleic acids comprises a standardized or a non-standardized set of nucleic acids; the second set of nucleic acids to comprises chimeric nucleic acid sequence fragments; the second set of nucleic acids is derived from the group consisting of: cultured microorganisms, uncultured microorganisms, complex biological mixtures, tissues, sera, pooled sera or tissues, multispecies consortia, fossilized or other nonliving biological remains, environmental isolates, soils, groundwaters, waste facilities, and deep-sea environments; the second set of nucleic acids is synthesized; the second set of nucleic acids is derived from the group consisting of: individual cDNA molecules, cloned sets of cDNAs, cDNA libraries, extracted RNAs, natural RNAs, in vitro transcribed RNAs, characterized genomic DNAs, uncharacterized genomic DNAs, cloned genomic DNAs, genomic DNA libraries, enzymatically fragmented DNAs, enzymatically fragmented RNAs, chemically fragmented DNAs, chemically fragmented RNAs, physically fragmented DNAs, and physically fragmented RNAs; the single-stranded nucleic acid templates each comprise at least one affinity-label; the method further comprises performing each step sequentially in a single reaction vessel. Additional preferred features of this embodiment appear elsewhere in this application.

A method for producing in vitro a plurality of polynucleotides having at least one desirable property, said method comprising: (a) subjecting a plurality of starting or parental polynucleotides to an exonuclease-mediated recombination process so as to produce a plurality of progeny polynucleotides; and (b) subjecting the progeny polynucleotides to an end selection-based screening and enrichment process, so as to select one or more of the progeny polynucleotides having at least one desirable property. Some preferred features of this alternative embodiment include: the recombination process generates ligation-compatible ends in the plurality of progeny polynucleotides; the method further comprises one or more intermolecular ligations between members of the progeny polynucleotides via the ligation-compatible ends, thereby achieving assembly and/or reassembly mutagenesis; and the intermolecular ligations are directional ligations. Additional preferred features of this embodiment appear elsewhere in this application.

A method for producing a plurality of mutant polypeptides having at least one desirable property, said method comprising: (a) subjecting a plurality of starting or parental polynucleotides to an exonuclease-mediated recombination process so as to produce a plurality of progeny polynucleotides; (b) introducing the progeny polynucleotides into a host cell so as to cause expression of a plurality of mutant polypeptides having an end selection marker; and (c) subjecting the mutant polypeptides to an end selection-based screening so as to select one or more having at least one desirable property. Some preferred features of this alternative embodiment include: the recombination introduces ligation-compatible ends into the progeny polynucleotides and wherein the method further comprises ligation of the progeny polynucleotides into an expression vector system via the ligation-compatible ends prior to introducing the progeny polynucleotides into the host cell; the method further comprises expression cloning of the polynucleotide set, and the screening involves screening of a plurality of the mutant polypeptides produced by the expression cloning. Other preferred features of this alternative embodiment appear elsewhere in this application.

A method of making a recombined nucleic acid that encodes a product having a desired property, the method comprising: (a) providing at least one single-stranded polynucleotide; (b) hybridizing a plurality of nucleic acid fragments to the single-stranded polynucleotide, which nucleic acid fragments are produced by fragmentation of a plurality of non-identical substrate nucleic acids; (c) extending and ligating the resulting hybridized nucleic acid fragments, thereby producing one or more recombined nucleic acid; and, (d) screening or selecting one or more product encoded by the recombined nucleic acid, or a complementary strand thereto, for the desired property, thereby identifying the recombined nucleic acid that encodes the product having the desired property. Preferred features of this alternative embodiment appear elsewhere in this application.

A method of identifying a recombined DNA molecule encoding a protein with a desired functional property, comprising: (a) providing at least one single-stranded uracil-containing DNA molecule, which single-stranded uracil-containing DNA molecule, or a complementary strand thereto, encodes a protein; (b) providing a plurality of non-identical DNA fragments capable of hybridizing to the single-stranded uracil-containing DNA molecule, wherein said DNA fragments are produced by fragmentation of one or more substrate DNA molecules encoding at least one additional variant of the protein and wherein the fragmentation is by digestion with DNAse I; (c) contacting the single-stranded uracil-containing DNA molecule with the plurality of DNA fragments, thereby producing an annealed DNA molecule; (d) incubating the annealed DNA molecule with a polymerase and a ligase, thereby producing a recombined DNA strand annealed to the uracil-containing DNA molecule; (e) amplifying the recombined DNA strand under conditions wherein the uracil-containing DNA molecule is not amplified, thereby producing a population of recombined DNA molecules; and, (f) screening or selecting the population of recombined DNA molecules to identify those that encode a polypeptide having the desired functional property, thereby identifying one or more DNA molecules(s) that encode a polypeptide with the desired functional property. Some preferred features of this alternative embodiment appear elsewhere in this application.

A method of producing a recombined polynucleotide having a desired characteristic, comprising: (a) providing a plurality of related-sequence double-stranded template polynucleotides, comprising polynucleotides with non-identical sequences; (b) providing a plurality of single-stranded nucleic acid fragments capable of hybridizing to the template polynucleotides; (c) hybridizing single-stranded nucleic acid fragments to the template polynucleotides and extending the hybridized fragments on the template polynucleotides with a polymerase, thereby forming a plurality of sequence-recombined polynucleotides; (d) subjecting the sequence recombined polynucleotides of step (c) to at least one additional cycle of recombination to produce further sequence-recombined poly-nucleotides; and, (e) selecting or screening the further sequence-recombined polynucleotides for the desired characteristic. Some preferred features of this alternative embodiment appear elsewhere in this application.

A method of non-stochastically producing a library of chimeric nucleic acid molecules having an overall assembly order that is non-random comprising:(a) non-randomly generating a plurality of nucleic acid building blocks having mutually compatible ligatable ends; and (b) assembling the nucleic acid building blocks, such that a designed overall assembly order is achieved; whereby a set of progenitor templates can be shuffled to generate a library of progeny polynucleotide molecules and correspondingly encoded polypeptides, and whereby screening of the progeny polynucleotide library provides a means to identify a desirable species that have a desirable property.

A method of non-stochastically producing a library comprised of a defined number of groupings comprised of one or more groupings of chimeric nucleic acid molecules having an overall assembly order that is chosen by design, said method comprised of-(a) generating by design for each grouping a set of specific nucleic acid building blocks having serviceable mutually compatible ligatable ends, and (b) assembling these nucleic acid building blocks according to said groupings, such that a designed overall assembly order is achieved; whereby a set of progenitor templates can be shuffled to generate a library of progeny polynucleotide molecules and correspondingly encoded polypeptides, and whereby the expression screening of the progeny polynucleotide library provides a means to identify a desirable species that has a desirable property.

### EXAMPLE I

The object of Example I is to produce recombinant polynucleotides from the kanamycin resistance gene, using non-identical fragments.

First, the resistance gene (1 Kb) of pACYC184 is cloned in the polylinker of M13 mp18 so that the non-identical phagemide contains the noncoding strand of the gene.

In parallel, this gene is amplified by PCR mutagenesis (error-prone PCR) with two initiators that are complementary to vector sequence M13 mp18 on each side of the gene sequence. The initiator for the noncoding strand is phosphorylated while the initiator for the coding strand is not. The product of the PCR mutagenesis is digested by the lambda exonuclease, which produces a library of coding strands for mutants of the kanamycin resistance gene.

This library of non-identical sequences is digested by a mixture of restriction enzymes, notably Hae III, Hinf I and Taq I. The resulting non-identical fragments are then hybridized with the non-identical phagemide and ligated with a thermostable ligase. This step is repeated several times until the small fragments can no longer be observed during deposition on an agarose gel. Meanwhile, the band corresponding to the non-identical of the complete resistance gene becomes a major component of the “smear” visible on the gel.

The band corresponding to the size of the gene is cut from the gel and purified. It is then hybridized with two complementary oligonucleotides (40 mer) of the M13 mp18 sequences on each side of the gene and this partial duplex is digested by Eco RI and Sph I, then ligated in an M13 mp18 vector digested by the same enzymes.

The cells transformed with the ligation product are screened for increased resistance to kanamycin.

The cloning of non-identical recombinant molecules can optionally be performed by PCR with two initiators of the complete gene and cloning of the double-stranded product of this amplification. To avoid undesirable mutations, this amplification should be performed with polymerase of the Pfu type and with a limited number of cycles.

The plasmids of the clones that are significantly more resistant to kanamycin than the initial stock are purified and used for PCR with the polymerase Pfu, under high fidelity conditions, with the phosphorylated/nonphosphorylated initiator couple as previously defined. This produces the second generation of non-identical fragments after a treatment with lambda exonuclease and fragmentation with restriction enzymes. The enzymes used for this step can comprise a different mixture (e.g., Bst NI, Taq I and Mnl I).

The recombination and selection steps are repeated several times until a substantial increase in resistance to kanamycin is obtained.

### EXAMPLE II

I. SUMMARY

The starting library included 10 gene mutants of ponB, coding for the PBPlb of E. coli (1). The sequence of each mutant differed from that of the native gene by a non-homologous zone 3-16 bases in length resulting from the substitution of five initial codons by five alanine codons, according to the technique described by Lefevre et al and incorporated herein (8).

The substitution represented a unique site of the restriction enzyme Pvu II surrounded by two Pst I enzyme sites, which permitted the mutants to be distinguished from each other by their digestion profile. FIG. 3 represents the positions of the ten zones of mutations (Pvu II and Pst I) carried by each mutant.

After PCR amplification of the mutants, the PCR products were purified and mixed in equimolar quantity in order to form the library. The polynucleotide sequences of this library were digested with the restriction enzymes Hinf I and Bsa I, in such a way as to generate libraries of restriction fragments. The restriction fragments were then incubated with various amounts of the wild-type template, at different quantities, in the presence of a thermostable ligase. After several denaturation/hybridization/ligation cycles, a fraction of the reaction mixture was used to carry out a PCR amplification with a couple of primers specific to the 5′ and 3′ ends of the mutant genes and non-specific to the 5′ and 3′ ends of the wild-type template. The amplification product was cloned and the clones were analyzed for their digestion profile with the Pvu II or Pst I restriction endonucleases. The obtained profiles indicated which fragments of the mutants were able to be recombined with the others to form an entire gene.

II. Materials and Methods

A. Strains and Plasmids

The strain MC1061 (F⁻ araD139, Δ (ara-leu)7696, galE15, galK16, Δ (lac)X74, rpsL (StrR), mcrA mcrB1, hsdR2 (rₖ⁻mₖ⁺)) is derived from Escherichia coli K12.

The vector pARAPONB stems from the vector pARA13 (3) in which the ponB gene carrying a thrombin-cutting site (9) was introduced between the restriction sites Nco I and Nar I. The vector pET26b+is one of the pET vectors developed by Studier and Moffatt (10) and commercialized by NOVAGEN Corporation.

B. Oligonucleotides

The oligonucleotides were synthesized by ISOPRIM corporation (Toulouse). The oligonucleotide sequences are reported in Table I below.

TABLE I

| | |
|---|---|
| Oligo N | 5′ ACTGACTACCATGGCCGGGAATGACCGCGAGCC 3′ |
| Oligo E | 5′ CCGCGGTGGAGCGAATTCTAATTACTACCAAACATATCC |
| | 3′ |
| Oligo M1 | 5′ GCGCCTGAATATTGCGGAGAAAAAGC 3′ |
| Oligo M2 | 5′ ACAACCAGATGAAAAGAAAGGGTTAATATC 3′ |
| Oligo A1 | 5′ ACTGACTACCATGGCC 3′ |
| Oligo A2 | 5′ CCGCGGTGGAGCGAATTC 3′ |

C. Reagents

The restriction and modification enzymes cited in Table II below were used according to the recommendations of the suppliers.

TABLE II

| Enzyme | Concentration | Supplier |
|---|---|---|
| NcoI | 10 U/μl | New England Biolabs |
| PstI | 20 U/μl | New England Biolabs |
| Eco RI | 20 u/μl | New England Biolabs |
| Bsa I | 5 U/μl | New England Biolabs |
| Hinf I | 10 U/μl | New England Biolabs |
| Pvu II | 10 U/μl | New England Biolabs |
| T4 DNA ligase | 400 U/μl | New England Biolabs |
| Taq DNA polymerase | 5 U/μl | PROMEGA |
| AMPLIGASE | 100 U/μl | EPICENTRE |

The buffers used are reported in Table III below.

TABLE III

| Buffers | Composition |
|---|---|
| T | Tris HCl 10 mM, pH 8.0 |
| Polymerization | Tris HCl 100 mM pH 8.3, MgCl₂ 15 mM, KCl 500 mM, |
| 20X | 1.0% TRITON X100 ® |
| Restriction | 500 mM NaCl, 100 mM Tris HCl pH 7.9, 100 mM |
| A 10X | MgCl₂, 10 Mm DTT, |
| Restriction | 1 M NaCl, 500 mM Tns HCl pH 7.9, 100 mM |
| B 10X | MgCl₂, 10 mM DTT |
| Restriction | 500 mM NaCl, 1 M Tris HCl pH 7.5, 100 mM mM |
| C 10X | MgCl₂, 0.25% TRITON X100 ® |
| AMPLIGASE | 200 mM Tris HCl pH 8.3, 250 mM KCl, 100 mM |
| 10X | MgCl₂, 5 mM NAD, 0.1% TRITON X100 ® |
| Ligation | 500 mM Tris HCl pH 7.5, 100 mM MgCl₂, |
| 10X | 100 mM DTT, 10 mM ATP, 250 μg/ml BSA |

III. Preparation of Template

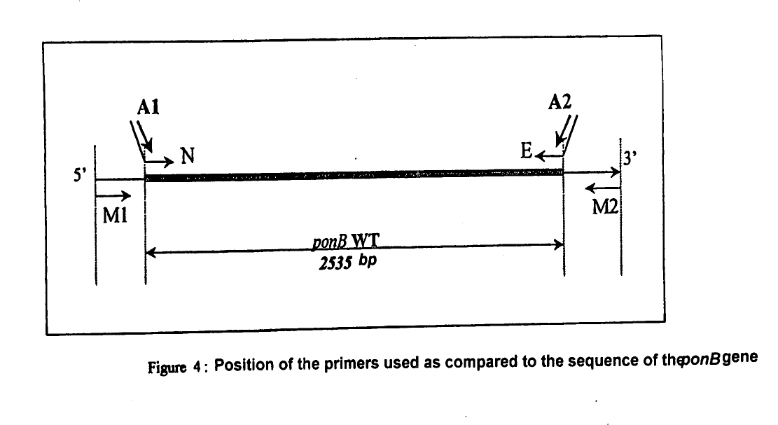

The wild type ponB gene was amplified by a PCR reaction step by using as primers the oligonucleotides M1 and M2 (FIG. 4). Five PCR reactions were prepared by adding 50 ng of pPONBPBR plasmid carrying the wild type gene (7) to a mixture containing 10 μJ of polymerization buffer, 10 μl of dNTPs 2 mM, 20 pmol of each oligonucleotide M1 and M2, and 5U of Taq DNA polymerase, in a final volume of 100 μl. These mixtures were incubated in Perkin-Elmer 9600 Thermocycler according to the following program: (94° C.−2 min.)−(94° C. 15 sec. −60° C. 30 sec. −72° C. 1 min.)×29 cycles−(72° C.−3 min.).

The product of the five PCR was mixed and loadedon a 1% TBEagarose gelAfter migration and staining of the gel with ethidium bromide, the band at 2651 bp, corresponding to the ponB gene amplification product surrounded by two fragments of 26 bp and 90 bp respectively, was visualized by trans-illumination under ultraviolet, and cut out with a scalpel in order to be purified with the QUIAquick system (QIAGEN). Allthe DNA thus purified was eluted in 120 μl of buffer T. The concentration of this DNA was approximatively 100 ng/μl as measured by its absorbanceat 260 nm.

IV. Preparation of the Library

A. Amplification of the Mutant Genes

The genes of the ten mutants were separately amplified by a PCR reaction witholigonucleotides N and E. These oligonucleotides introduce respectively the restriction sites Nco I and Eco RI, permitting the cloning of the products obtained with these two sites.

Each PCR reaction was prepared by adding 50 ng of the plasmid carrying the mutant gene to a mixture containing 10 μl of polymerization buffer, 10 μl of dNTPs 2 mM, 20 pmol of each oligonucleotide N and E, and 5U of Taq DNA polymerase, in a final volume of 100 μl. This mixture was incubated in a Perkin-Elmer 9600 thermocycler according to the following program: (94° C.−2 min.)−(94° C. 15 sec.−60° C. 30 sec.−72° C. 1 min.)×29 cycles−(72° C.−3 min.).

The specificity of the genetic amplification was verified by restriction profile with the Pvu II endonuclease, by incubating 5 μl of each PCR product 1 hour at 37° C. in a mixture containing 3 μl of restriction buffer A and 5U of the Pvu II enzyme in a final volume of 30 μl 15 μl of that digestion reaction were loaded on a TBE 1% agarose gel. After migration and staining with ethidium bromide, the gel was exposed to ultraviolet. The visualization of the restriction fragments permitted confirmation of the specificity of the genetic amplification of each mutant gene.

In parallel, 3 μl of each PCR reaction were loaded on a TBE 1% agarose gel. After migration, the gel was treated as above. The intensity of each band permitted the assessment that the genetic amplifications had the same yield.

B. Creation of Libraries of Restriction Fragments.

50 μl of each of the ten PCR were mixed and loaded on a 1% TBE agarose gel. After migration and staining with ethidium bromide, the band at 2572 bp, corresponding to the amplification product of the genes of the ten mutants, was cut out with a scalpel and purified with the Quiaquick system (QIAGEN). Allthe DNA thus purified was eluted in 120 μl of buffer T. The concentration of this DNA was approximately 100 ng/μl according to its absorbance at 260 nm.

In order to generate the libraries of restriction fragments, 100 μl of this DNA were incubated for one hour at 50° C. in a mixture containing 12 μl of restriction buffer B, 1.2 μl of BSA (at 10 mg/ml), 25 U of the enzyme Bsa I and 4 μl of water. Then, 2 μl of restriction buffer B, 2 μl of BSA (at 1 mg/ml), 50 U of the enzyme Hinf I and 11.5 μl of water were added to the mixture, which was incubated for one hour at 37° C. The digestion mixture was purified on a QIAquick column (QIAGEN), and eluted with 30 μl of buffer T. 1 μl of this eluate was loaded on a 1% TBE agarose gel in order to verify that the digestion had been total, and that it had generated 6 restriction fragments, and consequently six libraries of fragments, of 590 bp, 500 bp, 472 bp, 438 bp, 298 bp and 274 bp. The concentration of this DNA was approximately 250 ng/μl according to its absorbance at 260 nm.

V. Recombining Ligation Reaction (RLR)

The RLR reaction was carried out by incubating determined quantities of restriction fragments Hinf 1-Bsa I from the genes of ten mutants with the complete template (i.e., the wild type ponB gene), in the presence of a thermostable DNA ligase. The table IV below reports the composition of the mixtures for RLR.

TABLE IV

| | RLR 1 | RLR 2 | RLR 3 | RLR 4 | T- |
|---|---|---|---|---|---|
| Fragments Hinf I - Bsa I | 0.5 μl | 1 μl | 2 μl | 5 μl | 5 μl |
| of ten mutants | | | | | |
| (100 ng/μl) | | | | | |
| Wild type ponB template | 0.6 μl | 1.2 μl | 2.4 μl | 6 μl | 6 μl |
| (100 ng/μl) | | | | | |
| AMPLIGASE 10X Buffer | 2 μl | 2 μl | 2 μl | 2 μl | 2 μl |
| AMPLIGASE (25 U/μl) | 1 μl | 1 μl | 1 μl | 1 μl | — |
| H₂O | qsp 20 μl | qsp 20 μl | qsp 20 μl | qsp 20 μl | qsp 20 μl |

The negative control is identical to the reaction of RLR4, but does not contain thermostable DNA ligase. These different mixtures were covered with a drop of mineral oil and incubated in a Perkin-Elmer 9600 thermocycler in 200 μl microtubes according to the following program: (94° C., 5 min.)−(94° C., 1 min.−65° C., 4 min.)×35 cycles.

10 μl of each RLR reaction were then added to a PCR reaction mixture containing 10 μl of polymerization buffer, 10 μl of 2 mM dNTPs, 40 pmol of each oligonucleotide A1 and A2, and 5 U of Taq DNA polymerase in a final volume of 100 μl. This mixture was incubated in a Perkin-Elmer 9600 thermocycler according to the following program: (94° C., 5 min.)−(94° C., 30 sec.−46° C., 30 sec.−72° C., 1 min.)×29 cycles−(72° C., 2 min.). This PCR reaction permitted specific amplification of the ligation products formed in the course of the RLR reaction, without amplifying the template, since the oligonucleotides A1 and A2 are not able to hybridize with the template (it), as shown in FIG. 4.

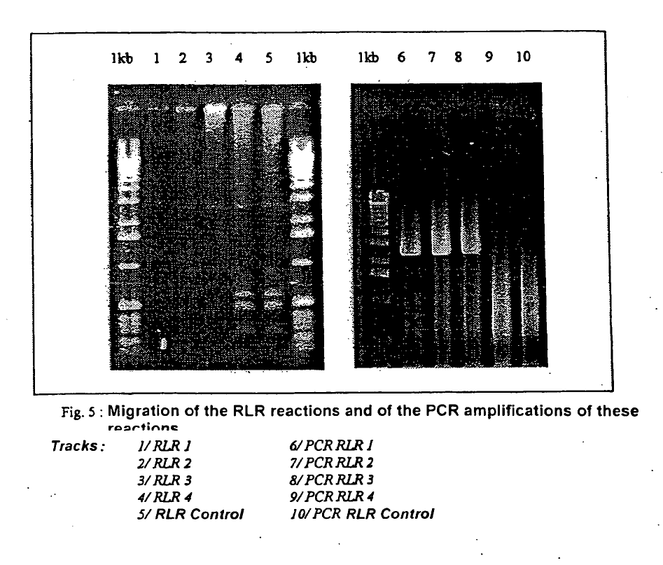

5 μl of each RLR reaction and 10 μl of each of the previous PCR reactions were loaded on a 1% TBE agarose gel. After staining with ethidium bromide, the gel was exposed to ultraviolet light, as shown in FIG. 5.

The analysis of this gel reveals that only the reaction of RLR4 contains, as the negative control, restriction fragments still visible (tracks 4 and 5).

The absence of PCR product for the negative control (track 10) reveals not only that the PCR reaction is specific (no amplification of the complete template), but also that the restriction fragments present in the mixture cannot be substituted for the primers to generate a contaminant PCR product under the chosen conditions. In parallel, the presence of a unique band at about 2500 bp in tracks 6, 7 and 8 demonstrates that an RLR product was able to be amplified by PCR for the RLR1, 2 and 3 reactions. These three RLR reactions therefore permitted the regeneration of one or more of the complete genes starting from six libraries of restriction fragments.

VI. Analysis of the Amplification Products

A. Cloning

The PCR amplification products of the RLR 1, 2 and 3 reactions were purified with the Wizard PCR Preps system (PROMEGA) and eluted in 45 μl of buffer T. 6 μl of each purified PCR were incubated 1 hour at 37° C. in a mixture containing 3 μl of restriction buffer C, 3 μl of BSA (1 mg/ml), 20 U of the Eco RI enzyme, 10 U of the Nco I enzyme and 15 μl of water.

In parallel, two vectors (pARAPONB and pET26b+) were prepared for the cloning. These vectors were linearized by incubating 3 μg of these plasmids for 2 hours at 37° C., in a mixture containing 3 μl of restriction buffer C, 3 μl of BSA (1 mg/ml), 20 U of the Eco RI enzyme, 10 U of the Nco I enzyme and 19 μl of water.

The linearized vectors as well as the digested PCR were purified on a TBE 1% agarose gel with the QIAquick system (QUIAGEN). Each vector or each digested PCR was eluted in 30 μl of buffer T.

The ligation of each PCR digested with each of the vectors was carried out according to the conditions described in table V below, and incubated at 16° C. for 16 hours.

TABLE V

| | Ligation with the vector | | | | | | | |
|---|---|---|---|---|---|---|---|---|
| | pARAPONB | | | | Ligation with the vector pET26b+ | | | |
| | LpAR1 | LpAR2 | LpAR3 | TlpAR | LpET1 | LpET2 | LpET3 | TLpET |
| PCR | 4 μl | — | — | — | 4 μl | — | — | — |
| amplification | | | | | | | | |
| RLR 1 digested | | | | | | | | |
| Nco I - Eco RI | | | | | | | | |
| PCR | — | 4 μl | — | — | — | 4 μl | — | — |
| amplification | | | | | | | | |
| RLR 2 digested | | | | | | | | |
| Nco I - Eco RI | | | | | | | | |
| PCR | — | — | 4 μl | — | — | — | 4 μl | — |
| amplification | | | | | | | | |
| RLR 3 digested | | | | | | | | |
| Nco I - EcoRI | | | | | | | | |
| Vector | 1 μl | 1 μl | 1 μl | 1 μl | — | — | — | — |
| pARAPONB | | | | | | | | |
| digested Nco I - | | | | | | | | |
| Eco RI | | | | | | | | |
| Vector pET26b+ | — | — | — | — | 1 μl | 1 μl | 1 μl | 1 μl |
| digested Nco I - | | | | | | | | |
| Eco RI | | | | | | | | |
| Ligation Buffer | 2 μl | 2 μl | 2 μl | 2 μl | 2 μl | 2 μl | 2 μl | 2 μl |
| Ligase | 1 μl | 1 μl | 1 μl | 1 μl | 1 μl | 1 μl | 1 μl | 1 μl |
| H₂O | 12 μl | 12 μl | 12 μl | 16 μl | 12 μl | 12 μl | 12 μl | 16 μl |

200 μl of chimiocompetent MC1061 cells (4) were transformed with 10 μL of each ligation by a thermal shock (5), and the cells thus transformed were spread over a selection medium.

No clone was obtained after transformation of ligation controls TLpAR and TLpET, thus indicating that the Nco 1-Eco RI vectors pARAPONB and pET26b+cannot undergo an intramolecular ligation.

B. Screening by PCR

A first screening of the clones obtained after transformation of the ligations with the vector pARAPONB was carried out by PCR. 42 colonies, 14 from each ligation LpAR1, LpAR2 and LpAR3, were resuspended individually in a PCR mixture containing 5 μl of polymerization buffer, 40 pmol of each oligonucleotide A1 and A2, 5 μl of 2 mM dNTPs and 5U of Taq DNA polymerase in a final volume of 50 μl. A negative control was obtained by adding to the PCR mixture 50 ng of the plasmid pBR322 in place of the colony. These 43 tubes were incubated in a Perkin-Elmer 9600 thermocycler according to the following program: (94° C., 5 min.)−(94° C., 30 sec.−46° C., 30 sec.−72° C., 1 min.)×29 cycles—(72° C., 2 min.). 5 μl of each of these PCR reactions were then incubated for 1 hour at 37° C. in a mixture containing 2 μl of restriction buffer A, 2 μl of BSA (1 mg/ml) and 5 U of the restriction enzyme Pvu II in a final volume of 20 μl.

10 μl of each of these digestions were loaded on a TBE 1% agarose gel in parallel with 5 μL1 of each non-digested PCR (thus avoiding possible confusion of non-specific bands of the PCR with a fragment obtained by restriction digestion). After migration and staining of this gel with ethidium bromide, the bands resulting from the digestion by the enzyme Pvu II were analyzed in order to determine which fragment(s) of initial mutants was/were associated with the others in order to reconstruct an entire gene. This screening reveals the presence of 27 genes carrying one mutation, 7 genes carrying two mutations and 8 genes no longer carrying any mutation.

C. Screening by Plasmidic DNA Minipreparation

The second screening was carried out by extracting the plasmidic DNA (5) from 21 clones resulting from the transformation of the ligations with the vector pET26b+(7 clones of each ligation). 5 μl of the plasmidic DNA thus obtained for each clone were incubated for 1 hour at 37° C. in a mixture containing 1 μl of restriction buffer C, 6 U of the enzyme Pst I, 3 U of the enzyme Nco 1 and 6 U of the enzyme Eco RI in a final volume of 10 μl. 5 μl of each of these digestions were loaded on a TBE 1% agarose gel. After migration and staining of this gel with ethidium bromide, the bands resulting from the digestion by the Pst I enzyme were analyzed in order to determine which fragment(s) of the initial mutants had associated with the others in order to reconstruct an entire gene. This screening reveals the presence of 13 genes carrying a mutation, 5 genes carrying two mutations and 3 genes no longer carrying a mutation.

D. Statistical Analysis of the Recombinations.

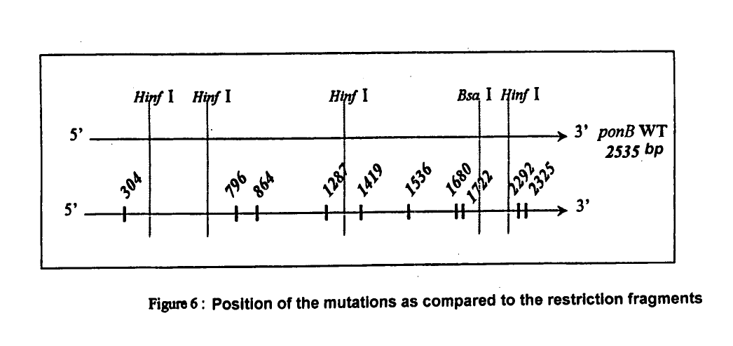

In view of the position of each mutation with regard to the cutting sites of the enzymes Hinf I and Bsa I (see FIG. 6), it is possible to calculate the probability of obtaining through RLR a gene carrying 0, 1, 2, 3, or 4 of the mutations of the initial genes.

Assuming that the RLR reaction is totally random, the probabilities P are as follows:

$$P ( 0 mutation ) = {\prod }_{i = 6}^{9} ( \frac{i}{10} ) = 30.24 \%$$

$$P ( 1 mutation ) = {\sum }_{n = 1}^{4} [ \frac{n}{10 - n} {\prod }_{i = 1}^{4} ( \frac{10 - i}{10} ) ] = 44.04 \%$$

$$P ( 2 mutations ) = {\sum }_{n = 1}^{4} [ {\sum }_{a = 1}^{4 - n} ( \frac{10 - a}{a} ) ( \frac{10 - ( a + n )}{a + n} ) {\prod }_{i = 1}^{4} ( \frac{i}{10} ) ] = 21.44 \%$$

$$P ( 3 mutations ) = {\sum }_{n = 1}^{4} [ ( \frac{10 - n}{n} ) {\prod }_{i = 1}^{4} ( \frac{i}{10} ) ] = 4.04 \%$$

$$P ( 4 mutations ) = {\prod }_{i = 1}^{4} ( \frac{i}{10} ) = 0.24 \%$$

The two screenings carried out give results close to these statistical predictions, as reported in table VI below, thus indicating that the RLR reaction is quasi-random. A slightly higher proportion of genes carrying one mutation, to the detriment of the genes carrying zero mutation, is observed. This phenomenon could be attributed to a weak toxicity of the ponB gene already observed and to the slight of expression leakage of vectors pARAPONB and pET26b+, which would favor the selection of genes carrying an inactivating mutation.

TABLE IV

| | % | | | | |
|---|---|---|---|---|---|
| | 0 | 1 | 2 | 3 | 4 |
| | mutation | mutation | mutations | mutations | mutations |
| Statistics | 30.24 | 44.04 | 21.44 | 4.04 | 0.24 |
| PCR | 21 | 63 | 16 | 0 | 0 |
| Screening | | | | | |
| Mini- | 14 | 62 | 24 | 0 | 0 |
| preparation | | | | | |
| Screening | | | | | |

### EXAMPLE III

Example III depicts an embodiment of the invention that employs controlled digestion.

I. Materials and Methods

A. Bacterial Strains, Genomic and Plasmid DNA

For all DNA manipulations, standard techniques and procedures were used. E coli MC1061DE3 cells were used to propagate the expression plasmid pET26b+(Novagen).

B. Oligonucleotides

All synthetic oligonucleotide primers for PCR were synthetized by MWG Biotech. The sense primer 5′ AGGAATTCCATATGCGAAAGAAAAGACGGGGA 3′ and the antisense primer 5′ ATAAAGCTTTCACTTGATGAGCCTGAGATTTC 3′ were used to amplify the Thermotoga Neapolitana Xylanase A gene and introduce NdeI and HindIII restriction sites (underlined). The NdeI site contained the initial codon (boldface).

C. Enzymes

Restriction enzymes, DNA polymerases and thermostable ligase were purchased from NEB and EPICENTRE and used as recommended by the manufacturers.

D. DNA Amplification, Cloning and Expression

PCR amplifications were carried out on a PE 9600 thermocycler. The Thermotoga Neapolitana Xylanase A amplicon was digested with primer-specific restriction endonucleases, ligated into compatible site on pET26b+, and transformed into E coli MC1061DE3. The MC1061DE3 clone containing the pET26b+XynA expression vector was propagated at 37° C. in LB containing kanamycin (60 μg/ml).

E. Biochemical Characterization

Thermal inactivation experiments were performed directly on E coli expressing XynA. Cells were re-suspended, after centrifugation at 6000 g for 5 min at 4° C., in 200 mM acetate buffer pH 5.6. Re-suspension was performed with an appropriate volume in order to standardize the amount of cell per sample. 150 μl of cells were then incubated at the appropriate temperature during different times. 100 μl of these cells were added to 100 μl of 0.5% (w/v) of xylan in 200 mM acetate buffer pH 5.6 and incubated 10 min at 80° C. Then, 200 μl of 3,5-Dinitrosalicylic acid were added and boiled 5 min, refrigerated 5 min on ice and centrifuged 5 min at 12000 g. 150 μl were transferred in μtiterplate and OD at 540 nm was measured.

For optimal temperature experiments, 100 μl of 0.5% (w/v) of xylan in 200 mM acetate buffer pH 5.6, were added to 100 μl of resuspended cells and incubated for 10 min at different temperatures during the 10 min. Then, 200 μl of 3,5-dinitrosalicylic acid were added and boiled for 5 min, refrigerated for 5 min on ice and centrifuged for 5 min at 12000 g. 150 μl were transferred in μtiterplate and OD at 540 nm was measured.

II. Results

A. Generation of Low Thermostable Mutant of XynA

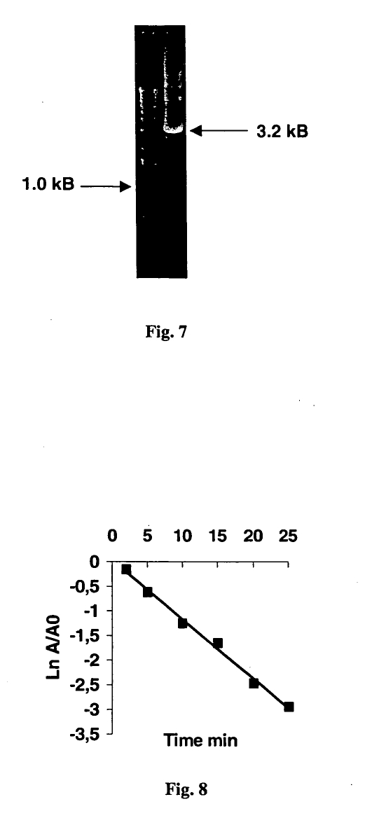

To generate a low thermostable mutant of XynA protein, error-prone PCR was performed as shown in FIG. 7, Error-prone PCR on WT XynA gene, using 1% agarose gel. The products were digested with primer-specific restriction endonucleases, ligated into compatible sites on pET26b+, and transformed into E coli MC1061DE3 to generate an error-prone library.

One clone (mutant 33) from the error-prone library seemed to have very low thermostability compared to the WT protein. A rapid biochemical analysis, including determination of an optimal temperature and thermal inactivation, was done and compared to the WT one. Regarding the optimal temperature, mutant 33 had an optimal temperature around 78° C. compared to the WT one (above 90° C.) but, for mutant 33 no residual activity was detected after 30 min incubation at 82° C. or 1 min at 95° C. and the inactivation constant calculated from FIG. 8, Thermal inactivation of mutant 33 at 82° C., was estimated at 0,120 min⁻¹ at 82° C. No or low thermal inactivation was detected for the WT protein at these temperatures.

B. Shuffling Experiments

The mutant 33 and WT genes were then recombined using L-Shuffling™ technology to generate mutants with different thermostabilities. Different mutants were expected: mutants with WT optimal temperature, mutants with lower thermostability than WT and mutants with higher thermostability than that of the mutant 33's optimal temperature.

1) Fragments Library

After PCR amplification of WT and mutant 33, the products were digested with a mix of six restriction enzymes, HincII, BamHI, XhoI, SphI, EcoRI, EcoRV, generating eight fragments (from 120 to 700 pb). See FIG. 9, Fragmentation of PCR products with a mix of six restriction endonucleases, using 3% agarose gel.

2) Shuffling Experiment

RLR (recombining ligation reaction) was performed with standardized fragments (shown in FIG. 9) and NdeI/HindIII digested pET26+XynA as template with the thermostable ligase using several cycles of denaturation and hybridation/ligation steps.

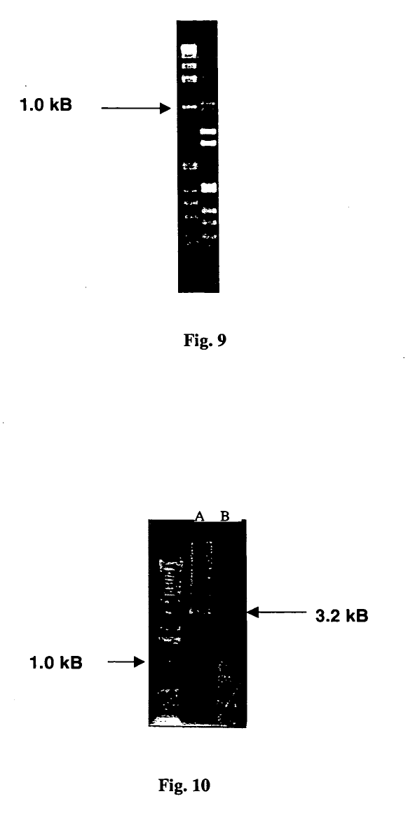

A negative control was done with the same conditions without the thermostable ligase (B) and the results are shown in FIG. 10, L-Shuffling™ experiments using 1% agarose gel. FIG. 10 shows that without thermostable ligase, the fragments are not used for any recombination. A selective digestion of the template was then performed by adding DpnI to the reaction mixture.

3) Cloning Products

A PCR Pfu amplification (FIG. 11, PCR Pfu on L-Shuffling™ products using 1% agarose gel) was performed on DpnI digested L-shuffling™ products both for A and B (negative control, FIG. 9) using 5′ sense and 5′ antisense synthetic primers and the protocol described above. No template amplification occurred, despite obtaining a large amount of amplified L-shuffling™ products for cloning. For this, L-shuffling/m products were digested with primer-specific restriction endonucleases, ligated into compatible sites on pET26b+, and transformed into E coli MC1061DE3 to generate a L-Shuffling™ library.

4) Biochemical Characterization

Several clones were selected from the L-Shuffling™ library for activity remaining after 30 min incubation at 82° C.

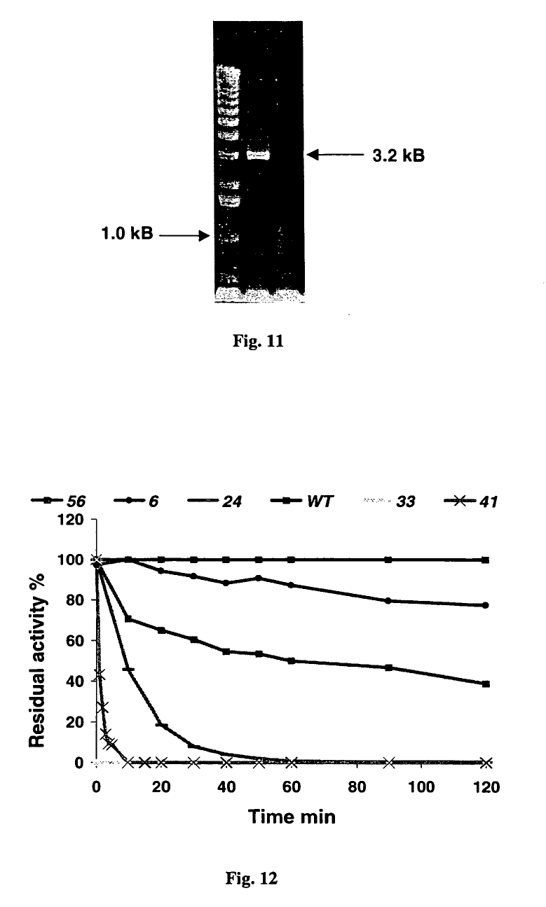

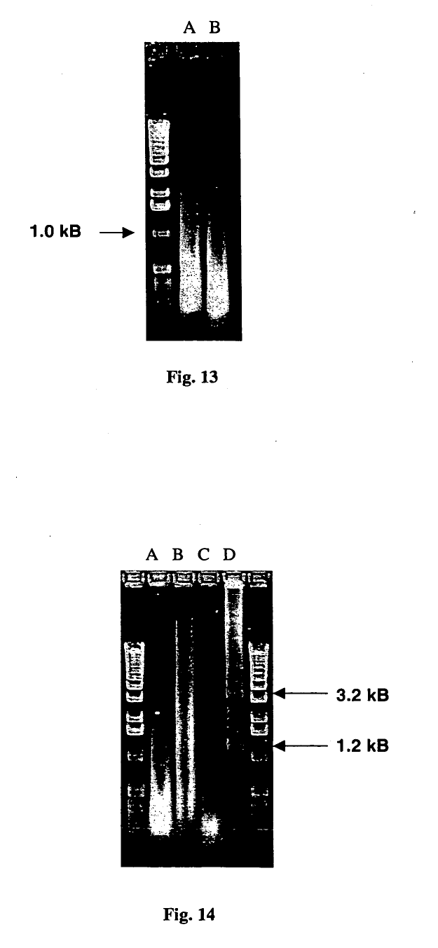

Clones 24, 41 and 56 (FIG. 12, Thermal inactivation of mutants at 95°) have the optimal temperature of mutant 33, and clone 6 has the optimal temperature of the WT xylanase. In these experimental conditions, WT xylanase retained 100% of activity after 120 min incubation at 95° C. On the contrary, for mutant 33 no residual activity was detected after 1 min at 95° C. FIG. 13 shows four mutants from the L-Shuffling™ library that exhibited characteristics that differ from those of the two parents.

### EXAMPLE IV

Example IV depicts an embodiment of the invention that employs random digestion.

I. Materials and Methods

A. Bacterial Strains, Genomic and Plasmid DNA

For all DNA manipulations, standard techniques and procedures were used. E coli MC1061DE3 cells were used to propagate the expression plasmid pET26b+(Novagen).

B. Oligonucleotides

All synthetic oligonucleotide primers for PCR were synthetized by MWG Biotech. The sense primer 5′ AGGAATTCCATATGCGAAAGAAAAGACGGGGA 3′ and the antisense primer 5′ ATAAAGCTTTCACTTGATGAGCCTGAGATTTC 3′ were used to amplify the Thermotoga Neapolitana Xylanase A gene and introduce NdeI and HindIII restriction sites (underlined). The sense primer 5′ GGAATTCCATATGGCGGCGGCAGCCGGCA 3′ and the antisense primer 5′ GGAATTCCTACTGCCGCTCCGATTGTGG 3′ were used to amplify the Acidobacterium capsulatum Xylanase gene and introduce NdeI and EcoRI restriction sites (underlined). The NdeI site contained the initial codon (boldface).

C. Enzymes

Restriction enzymes, DNA polymerases and thermostable ligase were purchased from NEB and EPICENTRE, and used as recommended by the manufacturers.

II. Results

The Thermotoga neapolitana gene (3.2 kB) and Acidobacterium capsulatum gene (1.2 kB) were recombined.

A. Fragments Library

PCR amplification on Thermotoga neapolitana and Acidobacterium capsulatum genes were performed, followed by digestion with DNaseI. See FIG. 13, DNaseI fragmentation of Thermotoga neapolitana (A) and Acidobacterium capsulatum (B) genes, using 1% agarose gel.

B. Shuffling Experiment

RLR was performed with standardized fragments (shown in FIG. 13) with thermostable ligase and thermostable flap, via several cycles of denaturation and hybridation/ligation.

Negative controls were performed under the same conditions but without the thermostable ligase and/or thermostable flap (A, B and C). The results are shown in FIG. 14, L-Shuffling™ experiments, using 1% agarose gel. FIG. 14 shows that without thermostable ligase and thermostable flap, the fragments are not recombined. In FIG. 14, A represents fragments without ligase and Flap activities; B represents fragments with only ligase; C represents fragments with only flap; and D represents the shuffling conditions.

### EXAMPLE V

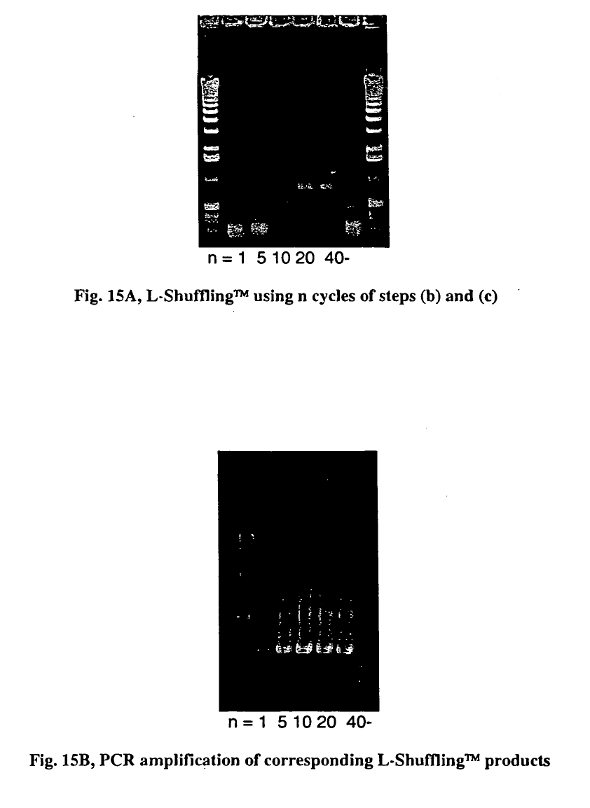

Example V employed the materials and methods of Example m but experimented with different numbers of cycles of steps (b) and (c). See FIG. 15A, L-Shuffling™ using n cycles of steps (b) and (c), and FIG. 15B, PCR amplification of corresponding L-Shuffling™ products. As shown in FIGS. 15A-B, at least one cycle (n=1) is necessary to obtain a recombinant polynucleotide.

### EXAMPLE VI

Example VI employed the materials and methods of Example III but experimented with seven quantities of fragments, as follows:

1: 1×

2: 2×

3: 3×

4: 4×

5: 11×

6: 14×

7: 17×

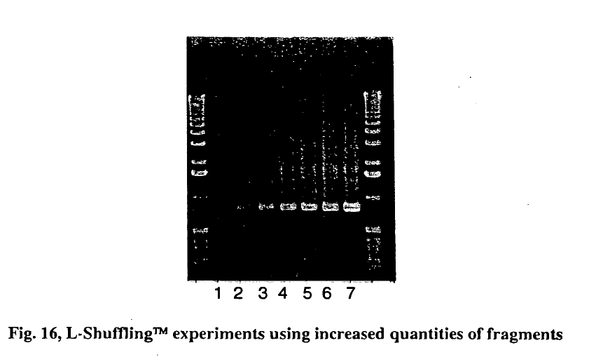

FIG. 16, L-Shuffling™ experiments using increased quantities of fragments, shows the results for these seven quantities.

The foregoing presentations are not intended to limit the scope of the invention. Although illustrative embodiments of the present invention have been described in detail and with reference to accompanying drawings, it is obvious to those skilled in the art that modifications to the methods described herein can be implemented. These and other various changes and embodiments may be effected by one skilled in the art without departing from the spirit and scope of the invention, which is intended to be determined by reference to the claims and their equivalents in light of the prior art.

### BIOGRAPHICAL REFERENCES

1) Broome-Smith J. K., Edelman Al, Yousif S. and Spratt B. G., (1985), The nucleotide sequence of the ponA and ponB genes encoding penicillin-binding proteins 1A and 1B of Escherichia coli K12, Eur. J. Biochem., 147, 437-446.

2) Caldwell R. C. and Joyce G., 1992, Randomization of genes by PCR mutagenesis, PCR Methods and Application, 2, 28-33.

3) Cagnon C., Valverde V. and Masson J.-M., (1991), A new family of sugar inducible expression vectors for Escherichia coli, Prot. Eng., 4, 843-847.

4) Hanahan D., (1985), Techniques for transformation of Escherichia coli, in DNA cloning: a practical approach, Glover D. M. (ed), IRL Press, Oxford vol 1,109-135.

5) Maniatis T., Fristch E. F. and Sambrook J., (1982), Molecular cloning. A laboratory Manual, Cold Spring Harbor Laboratory, Cold Spring Harbor, N.Y.

6) Landt et al., Gene, 96, 125-128, 1990.

7) Lefevre F., Topological Analysis of the Penicillin Binding Protein 1b of Escherichia coli, 1997, Thèse.

8) Lefevre F., Remy M. H. and Masson J. M., 1997 (a), Alanine-stretch scanning mutagenesis: a simple and efficient method to probe protein structure and function, Nuc. Acids Res., 25, 447-448.

9) Lefevre F., Remy M. H. and Masson J. M., 1997 (b), Topographical and functional investigation of Escherichia coli Penicillin-Binding Protein 1b by alanine stretch scanning mutagenesis, J. Bacteriol., 179, 4761-4767.

10) Studier F. W. and Moffatt B. A., 1986, Use of bacteriophage T7 RNA polymerase to direct selective high-level expression of cloned genes, J. Mol. Biol, 189, 113-130.

1-733. cancelled

734. A ligation-mediated in vitro method of recombining polynucleotides from a polynucleotide library, comprising:
fragmenting polynucleotides from the library;
hybridizing the fragments to an assembly template; and
ligating the hybridized fragments, wherein said fragments or said assembly template or a combination thereof optionally remain partially doubled stranded.

735. The method of claim 734, further comprising repeating the hybridizing step, before or after the ligating step, as necessary until ends of the hybridized fragments are substantially adjacent to each other on the assembly template, and ligating the adjacent ends to form recombined polynucleotide.

736. A ligation-mediated in vitro method of recombining polynucleotides from a polynucleotide library, comprising:
fragmenting polynucleotides from the library;
hybridizing the fragments with each other whereby one strand serves as an assembly template for another; extending the fragments by ligating the adjacent ends;
repeating the hybridizing step, before or after the ligating step, as necessary until ends of the hybridized fragments are substantially adjacent to each other on the assembly template; and
ligating the adjacent ends to form at least one recombined polynucleotides.

737. A method of ligase mediated shuffling polynucleotides, comprising:
conducting multiple cycles of denaturation, annealing and extenion on partially annealed polynucleotide strands having sequences from a plurality of polynucleotide variants under conditions whereby one strand serves as a template for extension of another strand with which it is partially annealed to generate a population of shuffled polynucleotides; and
screening or selecting the shuffled polynucleotides to identify a shuffled polynucleotide having a desired functional property;
wherein the cycles of denaturation are performed at 80-100 C.

738. A method of ligase-mediated shuffling polynucleotides, comprising:
conducting multiple cycles of denaturation, annealing and extenion on partially annealed polynucleotide strands having sequences from a plurality of polynucleotide variants under conditions whereby one strand serves as a template for extension of another strand with which it is partially annealed to generate a population of shuffled polynucleotides; and
screening or selecting the shuffled polynucleotides to identify a shuffled polynucleotide having a desired functional property;
wherein the annealing is performed at 40-65 C.

739. A method of ligase-mediated shuffling polynucleotides, comprising:
conducting multiple cycles of denaturation, annealing and extenion on partially annealed polynucleotide strands having sequences from a plurality of polynucleotide variants under conditions whereby one strand serves as a template for extension of another strand with which it is partially annealed to generate a population of shuffled polynucleotides; and
screening or selecting the shuffled polynucleotides to identify a shuffled polynucleotide having a desired functional property;
wherein the shuffled polynucleotides have a length of 500-50 kb.

740. A method of ligase-mediated shuffling polynucleotides, comprising:
conducting multiple cycles of denaturation, annealing and extenion on partially annealed polynucleotide strands having sequences from a plurality of polynucleotide variants under conditions whereby one strand serves as a template for extension of another strand with which it is partially annealed to generate a population of shuffled polynucleotides; and
screening or selecting the shuffled polynucleotides to identify a shuffled polynucleotide having a desired functional property;
wherein sequence are from polynucleotide variants of unknown sequence.

741. A method of ligase-mediated shuffling polynucleotides, comprising:
randomly cleaving a mixed population of polynucleotide vanants to produce fragments;
conducting multiple cycles of denaturation, annealing and extension on the fragments under conditions whereby one strand of a fragment serves as a template for extension of a strand from another fragment to generate a population of shuffled polynucleotides; and
screening or selecting the shuffled polynucleotides to identify a shuffled polynucleotide having a desired functional property.

742. A ligation-mediated in vitro method of recombining polynucleotides from a polynucleotide library, comprising:
fragmenting polynuclcotides from the library;
hybridizing at least partially the fragments to an assembly template; and
ligating the hybridized fragments.

743. The method of claim 742, further comprising repeating the hybridizing step, before or after the ligating step, as necessary until ends of the hybridized fragments are substantially adjacent to each other on the assembly template, and ligating the adjacent ends to forn recombined polynucleotide.

744. A ligation-mediated in vitro method of recombining polynucleotides from a polynucleotide library, comprising:
(i) providing single-stranded fragments, wherein at least one of the DNA fragments differs from at least one of the single-stranded polynucleotides in at least one nucleotide;
(ii) fragmenting the DNA substrate molecules to provide a mixture of fragmented substrate molecules that are capable of annealing to the single-stranded polynucleotides;
(iii) contacting the single-stranded polynucleotides with the mixture of fragmented substrate molecules to provide annealed nucleic acids; and
(iv) contacting the annealed nucleic acids with a polymerase, a ligase, or both a polymerase and a ligase to provide the library of variants of the DNA substrate molecules.

745. A combinatorial gene expression library, comprising a pool of expression constructs, each expression construct containing recombinant DNA wherein recombinant DNA are issued from the ligation template-mediated of fragments derived from a plurality of species of donor organisms.

746. A method of recombining homologous nucleic acids, the method comprising:
(i) hybridizing a set of family gene shuffling oligonucleotides on an assembling template; and,
(ii) elongating the set of family gene shuffling oligonucleotides, thereby providing a population of recombined nucleic acids.

747. The method of claim 746, wherein the elongating step is performed with a ligase.

748. The method of claim 746, the method further comprising:
(iii) denaturing the population of recombined nucleic acids, thereby providing denatured recombined nucleic acids;
(iv) reannealing the denatured recombined nucleic acids;
(v) extending by ligation the resulting reannealed recombined nucleic acids; and, optionally:
(vi) selecting one or more of the resulting recombined nucleic acids for a desired property.

749. The method of claim 746, wherein the set of family shuffling oligonucleotides comprise a plurality of codon-varied oligonucleotides

750. A method of identifying a recombinant nucleic acid with a desired property, the method comprising:
(a) providing a plurality of random fragments of at least a first and a second nucleic acid;
(b) recombining the random fragments one or more times to produce at least one recombinant nucleic acid; and,
(c) identifying at least one recombinant nucleic acid with the desired property.

751. A method for evolving a protein encoded by a DNA substrate molecule comprising:
(a) digesting at least a first and second DNA substrate molecule, wherein the at least a first and second substrate molecules differ from each other in at least one nucleotide, with a restriction endonuclease;
(b) ligating the mixture to generate a library of recombinant DNA molecules;
(c) screening or selecting the products of (b) for a desired property; and
(d) recovering a recombinant DNA substrate molecule encoding an evolved protein.

752. A composition, comprising: a set of nucleic acids, comprising:
a first subset of chemically synthesized oligonucleotide members which collectively correspond to at least a substantial portion of a first target nucleic acid; and, a second subset of chemically synthesized oligonucleotide members which collectively correspond to at least a substantial portion of a second target nucleic acid;
wherein the first and second target nucleic acids encode non-identical proteins and comprise a plurality of regions of difference, and wherein the first and second subsets of chemically synthesized oligonucleotide members correspond to the regions of difference, and the first and second subsets are present in substantially non-equimolar amounts.

753. A ligase-mediated on an assembling template method of recombining an oligonucleotide set, the method comprising:
aligning a plurality of homologous nucleic acid sequences to identify one or more regions of sequence heterogeneity;
synthesizing a plurality of different oligonucleotide member types which correspond to one of the regions of heterogeneity;
mixing the plurality of different oligonucleotide member types, thereby providing a set of oligonucleotides which comprise a plurality of different oligonucleotide members which comprise the at least one regions of sequence heterogeneity which corresponds to one or more of the regions of heterogeneity in the plurality of homologous nucleic acid sequences; and,
recombining one or more member of the oligonucleotide set with one or more nucleic acid corresponding to one or more of the homologous nucleic acid sequences.

754. A method of recombining an oligonucleotide set, the method comprising:
aligning a plurality of homologous nucleic acid sequences to identify one or more regions of sequence heterogeneity;
synthesizing a plurality of different oligonucleotide member types which correspond to one of the regions of heterogeneity; mixing the plurality of different oligonucleotide member types, thereby providing a set of oligonucleotides which comprise a plurality of different oligonucleotide members which comprise the at least one regions of sequence heterogeneity which corresponds to one or more of the regions of heterogeneity in the plurality of homologous nucleic acid sequences; and,
recombining one or more member of the oligonucleotide set with one or more nucleic acid corresponding to one or more of the homologous nucleic acid sequences.

755. A ligase-mediated method of replicating a template polynucleotide, comprising the ordered steps of:
providing overlapping fragments of a template polynucleotide by cleaving the template polynucleotide;
denaturing the fragments; conducting a multicyclic polynucleotide extension reaction on the denatured fragments in the absence of intact template to generate products comprising the template polynucleotide and/or variants thereof.

756. The method of claim 755, wherein the template polynucleotide is a whole genome.

757. A ligase-mediated method of replicating a template polynucleotide, comprising the ordered steps of:
providing overlapping fragments of a template polynucleotide;
denaturing the fragments; conducting a multicyclic polynucleotide extension reaction on the denatured fragments to generate products comprising the template polynucleotide and variants thereof;
screening or selecting the variants for a desired functional property.

758. The method of claim 757, wherein the template polynucleotide is a whole genome.

759. A ligase-mediated method of replicating a template polynucleotide, comprising:
obtaining a degraded template polynucleotide from nature;
cleaving the degraded template polynucleotide to produce fragments;
denaturing the fragments; conducting a multicyclic polynucleotide extension reaction on the denatured fragments to generate products comprising the template polynucleotide and/or variants thereof,

760. The method of claim 759, wherein the template polynucleotide is a whole genome.

761. A method of identifying variant with at least one desired property, the method comprising:
(a) providing a mixture of nucleic acid subsequences of two or more parental polynucleotides, wherein each parental polynucleotide differs from at least one other parental polynucleotide in at least one nucleotide;
(b) extending one or more of the nucleic acid subsequences with at least one ligase template-mediated to produce one or more recombined polynucleotides;
(c) expressing the one or more recombined polynucleotides;
(d) screening or selecting the one or more variants to identify at least variant with the at least one desired property;
(e) recovering at least one recombined polynucleotide encoding the at least variant identified in step (d); and,
(f) repeating (a)-(d) using the at least one recombined polynucleotide recovered in step (e) as at least one of the two or more parental polynucleotides of a repeated step (a).

762. A method of non-stochastically producing a library of chimeric nucleic acid molecules having an assembly order chosen by design, which method is comprised of:
(a) generating by design a plurality of specific synthetic nucleic acid building blocks having mutually compatible ligatable ends, and
(b) assembling the nucleic acid building blocks, such that a designed overall assembly order is achieved.

763. A method for producing a mutagenized progeny polynucleotide, comprising:
(a) subjecting a starting or parental polynucleotide set to an in vitro exonuclease- mediated reassembly process so as to produce a progeny polynucleotide set;
whereby the exonuclease-mediated reassembly process is exemplified, in a non-limiting fashion, by subjection to a 3′ exonuclease treatment, such as treatment with exonuclease IIL which acts on 3′ underhangs and blunt ends, to liberate 3′-termiinal but not 5′-terninal nucleotides from a starting double stranded polynucleotide, leaving a remaining strand that is partially or completely free of its original partner so that, if desired, the remaining strand may be used to achieve hybridization to another partner;
whereby the exonuclease-mediated reassembly process is further exemplified, in a non- limiting fashion, by subjection to a 5′ exonuclease treatment, such as treatment with red alpha gene product, that acts on 5′ underhangs to liberate 5′-temiinal nucleotides from a starting double stranded polynucleotide, leaving a remaining strand that is partially or completely free of its original partner so that, if desired, the remaining strand may be used to achieve hybridization to another partner;
whereby the exonuclease-mediated reassembly process is further exemplified, in a non- limiting fashion, by subjection to an exonuclease treatment, such as treatment with Mung Bean Nuclease or treatment with S INuclease or treatment with E. coli DNA Polymerase, that acts on overhanging ends, including on unhybridized ends, to liberate terminal nucleotides from an unhybridized single-stranded end of an annealed nucleic acid strand in a heteromeric nucleic acid complex, leaving a shortened but hybridized end to facilitate polymerase-based extension and/or ligase-mediated ligation of the treated end;
and whereby the exonuclease-mediated reassembly process is also exemplified by a dual treatment, that can be performed, for example, non-simultaneously, with both an exonuclease that liberates terminal nucleotides from underhanging ends or blunt ends as well as an exonuclease that liberates terminal nucleotides from overhanging ends such as unhybridized ends.

764. A method for producing and isolating a polypeptide having at least one desirable property comprised of the steps of:
(a) subjecting a starting or parental polynucleotide set to an exonuclease-mediated recombination process so as to produce a progeny polynucleotide set; and
(b) subjecting the progeny polynucleotide set to an end selection-based screening and enrichment process, so as to select for a desirable subset of the progeny polynucleotide set;
whereby the above steps can be performed iteratively and in any order and in combination,
whereby the end selection-based process creates ligation-compatible ends, whereby the creation of ligation-ompatible ends is optionally used to facilitate one or more intermolecular ligations, that are preferably directional ligations, within members of the progeny polynucleotide set so as to achieve assembly and/or reassembly mutagenesis,
whereby the creation of ligation-compatible ends serves to facilitate ligation of the progeny polynucleotide set into an expression vector system and expression cloning,
whereby the expression cloning of the progeny polynucleotide set serves to generate a polypeptide set,
whereby the generated polypeptide set can be subjected to an expression screening process, and
whereby expression screening of the progeny polypeptide set provides a means to identify a desirable species, e.g. a mutant polypeptide or alternatively a polypeptide fragment, that has a desirable property, such as a specific enzymatic activity.

765. A ligase-mediated assembling template mediate method for producing a recombined progeny polynucleotide, comprising subjecting a starting or parental polynucleotide set to an in vitro exonuclease-mediated reassembly process so as to produce a progeny polynucleotide set

766. A method of evolving a polynucleotide toward a desired lo functional property comprising:
(a) providing a plurality of polynucleotides comprising two or more species variants;
(b) shuffling said plurality of polynucleotides to form a population of recombinant polynucleotides;
(c) selecting or screening said population of recombinant polynucleotides for recombinant polynucleotides that have evolved toward the desired functional property,
(d) repeating steps (b) and (c) with the plurality of polynucleotides in step (b) comprising the recombinant 20 polynucleotides selected or screened in step (c) until a recombinant polynucleotide is obtained which has acquired the desired functional property, wherein at least one shuffling cycle comprises conducting a multi-cyclic polynucleotide extension process on partially annealed polynucleotide strands having sequences from the plurality of polynucleotides, the plurality of polynucleotides having regions of similarity and regions of heterology with each other and the partially annealed polynucleotide strands being partially annealed through the regions of similarity, under conditions whereby one strand serves as a template for extension of another strand with which it is partially annealed to generate said recombinant polynucleotides.

767. A method of generating chimeric nucleic acids, the method comprising:
hybridizing a first plurality of first parental single-stranded nucleic acids and a second plurality of second parental single-stranded nucleic acids, wherein the hybridized first and second parental single-stranded nucleic acids comprise at least one nonhybridized region of sequence diversity;
nicking at least one strand in the at least one nonhybridized region of sequence diversity;
cleaving the at least one nicked strand in the at least one nonhybridized region of sequence diversity to provide at least one sequence gap between hybridized regions; and, elongating, ligating, or both, the at least one sequence gap between the hybridized regions to generate chimeric progeny nucleic acids.

768. A method of combinatorially assembling nucleic acids, the method comprising: hybridizing at least two sets of nucleic acids, wherein a first of the at least to sets of nucleic acids comprises single-stranded nucleic acid templates and a second set of the at least two sets of nucleic acids comprises at least one set of nucleic acid fragments, which fragments hybridize to a plurality of subsequences on at least one member of the first set of nucleic acids, wherein hybridization of the first and second set of nucleic acids directs combinatorial assembly of a third set nucleic acids.

769. A method of producing recombinant oligonucleotides from two or more parent oligonucleotides by an in vitro-in vivo recombination method comprising the steps of:
specifying one or more selected cut points for each parent oligonucleotide;
preparing synthetic polymer fragments having sequences corresponding to the sequences of parent oligonucleotides that are cut at specified cut points;
extending the sequence of each fragment at a cut point against a parental template tc produce a set of oligonucleotide duplexes representing different combinations of fragments
(i) removing parent homoduplex oligonucleotides; and
providing a set of recombinants from the resulting heteroduplex oligonucleotides.

770. A method for producing a mutant polynucleotide encoding at least one desirable property, the method comprising:
(a) subjecting a plurality of first polynucleotides to simultaneous mutagenesis so as to produce a plurality of progeny polynucleotides, wherein the mutagenesis comprises subjecting a codon-containing template polynucleotide to amplification using a degenerate oligonucleotide for each codon to be mutagenized, wherein the degenerate oligonucleotide comprises a first homologous sequence and a degenerate triplet sequence, and
(b) subjecting the progeny polynucleotides to an end selection-based screening and enrichment process that creates ligation-compatible ends, so as to select one or more progeny polynucleotides encoding at least one desirable property.

771. A method of evolving a polynucleotide toward a desired functional property comprising:
(a) providing a plurality of polynucleotides comprising two or more species variants;
(b) shuffling said plurality of polynucleotides to form a population of recombinant polynucleotides;
(c) selecting or screening said population of recombinant polynucleotides for recombinant polynucleotides that have evolved toward the desired functional property,
(d) repeating steps (b) and (c) with the plurality of polynucleotides in step (b) comprising the recombinant polynucleotides selected or screened in step (c) until a recombinant polynucleotide is obtained which has acquired the desired functional property, wherein at least one shuffling cycle comprises conducting a mult-yclic polynucleotide extension process on partially annealed polynucleotide strands having sequences from the plurality of polynucleotides, the plurality of polynucleotides having regions of similarity and regions of heterology with each other and the partially annealed polynucleotide strands being partially annealed through the regions of similarity, under conditions whereby one strand serves as a template for extension of another strand with which it is partially annealed to generate said recombinant polynucleotides.

772. A method of shuffling polynucleotides, comprising:
conducting a polynucleotide extension process on overlapping segments having sequences of a population of variants of a polynucleotide encoding a plurality of genes under conditions whereby one segment serves as a template for extension of another segment to generate a population of recombinant polynucleotides at least one of which encodes the plurality of genes; and
screening or selecting recombinant polynucleotides encoding the plurality of genes to identify a recombinant polynucleotide encoding the plurality of genes having a desired functional property conferred by the genes or their expression products.

773. A method for forming at least one chireric polynucleotide comprising;
contacting a single-stranded polynucleotide template with a random population of oligonucleotides, under conditions wherein at least two oligonucleotides hybridize to the template; and
treating the hybridized oligonucleotides such that a chimeric polynudleotide is formed

774. A method for forming a chimeric polynucleotide comprising;
contacting a single-stranded polynucleotide template with a population of oligonucleotides under conditions such that at least two oligonucleotides hybridize to a given template, and wherein the population of oligonucleotides comprises oligonucleotides such that two or more regions of the template are complementary to two or more oligonucleotides of the population; and
ligating the hybridized oligonucleotides such that one chimeric polynucleotide, is generated.

775. A method for forming at least one chimeric polynucleotide comprising:
contacting a single-stranded polynucleotide template with a population of oligonucleotides, wherein at least two of the oligonucleotides hybridize to the same template, such that at least one flap is formed;
removing at least one flap; and
ligating the hybridized oligonucleotides such that one chimeric polynucleotide, is generated.

776. A method for forming a chimeric polynucleotide comprising:
contacting a single-stranded template with a population of oligonucleotides, under conditions such that at least two of the oligonucleotides hybridize to the template;
filling in gaps between the hybridized oligonucleotides; and
ligating the hybridized oligonucleotides such that a chimeric polynucleotide is formed.

777. A method for forming a chimeric polynucleotide comprising the following steps:
contacting a single-stranded template with a population of oligonucleotides produced by fragmenting a single-stranded nucleic acid or by chemical synthesis, under conditions such that at least two of the oligonucleotides hybridize to the template; and
ligating the hybridized oligonucleotides, thereby forming a template-length chimeric polynucleotide.

778. A method for forming a plurality of chimeric polynucleotides on single-stranded polynucleotide templates, wherein the number of chimeric polynucleotides formed and the number of single-stranded templates is in a ratio of about 1 comprising the following steps:
contacting a single-stranded template with a population of oligonucleotides produced by fragmenting a single-stranded nucleic acid or by chemical synthesis, under conditions such that at least two of the oligonucleotides hybridize to the template; and
ligating the hybridized oligonucleotides, thereby forming a template-length chimeric polynucleotide.

779. A method for forming a chimeric polynucleotide comprising:
preparing a single-stranded template comprising RNA;
contacting the single-stranded template with a population of oligonucleotides, under conditions such that at least two of the oligonucleotides hybridize to the template; and
treating the hybridized oligonucleotides such that at least one contiguous chimeric polynucleotide is formed.

780. A method for forming a chimeric polynucleotide comprising:
preparing a single-stranded polynucleotide template containing a plurality of uracil residues;
contacting the template with a population of oligonucleotides, wherein at least two of the oligonucleotides hybridize to the template;
treating the template with an enzyme;
rflling in gaps between hybridized oligonucleotides on the template; and
ligating adjacently hybridized oligonucleotides to form the chimeric polynucleotide.

781. A method for forming a chemically modified single-stranded polynucleotide template for use in a method of directed evolution comprising:
preparing a double-stranded polynucleotide comprising the single-stranded polynucleotide template and a complementary polynucleotide strand for each double-stranded nucleic acid a 5′ strand without a phosphate group; annealing the 5′ strands to form heteroduplex nucleic acids;
treating the heteroduplex nucleic acids with an enzyme that cleaves mismatches to yield homoduplexes; and denaturing the double-stranded polynucleotide;
adding a single-stranded oligonucleotide capable of annealing to the strand complementary to the single-stranded template;
and isolating the single-stranded polynucleotide template from its complementary strand and from the added oligonucleotide, thus yielding the purified single-stranded polynucleotide template.

782. A method for generating a chimeric polynucleotide, wherein one or more characteristics of the chimeric molecule is altered in comparison to a reference polynucleotide comprising the steps of:
preparing a double-stranded poly-nucleotide comprising a single-stranded polynucleotide template and a complementary polynucleotide strand, denaturing the double-stranded polynucleotide;
adding a single-stranded oligonucleotide capable of annealing to the strand complementary to the single-stranded template;
isolating the single-stranded polynucleotide template from its complementary strand and from the added oligonucleotide, thus yielding the purified single-stranded polynucleotide template; contacting the single-stranded template with a population of oligonucleotides, under conditions such that at least two of the oligonucleotides hybridize to the template;
trimming flaps;
filling in gaps;
ligating hybridized oligonucleotides to form at least one chimeric polynucleotide; selectively amplifying the chimeric polynucleotide with respect to the single-stranded polynucleotide template; and
selecting or screening the chimeric polynucleotide, wherein a characteristic is altered in comparison to the reference polynucleotide.

783. A method for forming a chemically modified single-stranded polynucleotide template for use in a method of directed evolution comprising:
preparing a double-stranded polynucleotide comprising the chemically modified single- stranded polynucleotide template and a complementary polynucleotide strand;
denaturing the double-stranded polynucleotide; and
isolating the single-stranded polynucleotide template from its complementary strand, thus yielding the purified single-stranded polynucleotide template.

784. A method for forming and selecting at least one chimeric polynucleotide, wherein one or more characteristics of the chimeric molecule is altered in comparison to a reference polynucleotide, comprising the steps of:
contacting a least one single-stranded polynucleotide template with a population of oligonucleotides under conditions wherein at least two oligonucleotides hybridize to the template;
treating hybridized oligonucleotides to form one chimeric polynucleotide hybridized to a template;
selectively amplifying the chimeric polynucleotide with respect to the templates; and
selecting or screening at least one chimeric polynucleotide, wherein the specified characteristic is altered in comparison to the reference polynucleotide.

785. A method for forming a chimeric polynucleotide comprising:
preparing a random population of oligonucleotides from at least one nucleic acid with a preselected nucleotide sequence;
contacting a single-stranded template with the population of oligonucleotides under conditions such that at least two of the oligonucleotides hybridize to the template; and
ligating the hybridized oligonucleotides such that a chimeric polynucleotide is formed.

786. A method for generating a mutagenized progeny polynucleotide from a collection of progenitor polynucleotides, comprising:
a) annealing a poly-binding nucleic acid strand to two mono-binding nucleic acid strands to generate an annealed heteromeric complex of nucleic acid strands, wherein the poly-binding nucleic acid strand and the two mono-binding nucleic acid strands are each derived from a different molecular species in said collection of progenitor polynucleotides;
b) and subjecting the unhybridized single-stranded ends of the annealed mono-binding nucleic acid strands in the heteromeric complex to an exonuclease treatment that degrades said unhybridized ends.
